(19) United States
(12) Patent Application Publication
(10) Pub. No.: US 2026/0218151 A1
(43) Pub. Date: Jul. 30, 2026

(54) METHOD OF DELIVERING NUCLEIC ACID TO T CELLS AND COMPOSITIONS FOR USE THEREOF

(71) Applicant: NanoVation Therapeutics Inc., Vancouver (CA)

(72) Inventors: Martin Attwood, Oxford (GB); Daniel Kurek, Vancouver (CA); Jessica A.F.D. Silva, Vancouver (CA); Jayesh Kulkarni, Vancouver (CA)

(21) Appl. No.: 19/372,824

(22) Filed: Oct. 29, 2025

Related U.S. Application Data
(63) Continuation of application No. PCT/CA2025/050523, filed on Apr. 10, 2025.
(60) Provisional application No. 63/632,601, filed on Apr. 11, 2024.
(60) Provisional application No. 63/680,680, filed on Aug. 8, 2024.

(51) Int. Cl. C12N 9/22; A61K 9/16; C12N 5/0783; C12N 15/11; C12N 15/88

(52) U.S. Cl. CPC C12N 9/226; A61K 9/1641; C12N 5/0636; C12N 15/111; C12N 15/88; C12N 2310/20; C12N 2510/00

(57) ABSTRACT

The present disclosure provides a method for delivery of nucleic acid to a T cell to produce a modified T cell, the method comprising contacting a lipid nanoparticle encapsulating the nucleic acid with the T cell ex vivo or in vivo, thereby causing cellular uptake of the nucleic acid, the lipid nanoparticle having between 30 mol % and 70 mol % of a neutral or zwitterionic amphipathic lipid having a net-neutral charge at physiological pH, an ionizable cationic lipid, and a sterol, wherein the lipid nanoparticle is substantially uncharged at physiological pH and has an apparent pKa of between 6.0 and 7.5, wherein the nucleic acid modifies the T cell to provide a T cell having a therapeutic, prophylactic or ameliorative effect in vivo. Further provided are compositions for use thereof and T cell preparations.

This application is a bypass continuation of PCT Patent Application No. PCT/CA2025/050523 having an international filing date of Apr. 10, 2025, which claims the priority benefit of U.S. Provisional Patent Application No. 63/680,680 filed on Aug. 8, 2024, and U.S. Provisional Patent Application No. 63/632,601 filed on Apr. 11, 2024, which are each incorporated herein by reference in their entirety.

### TECHNICAL FIELD

The present disclosure relates to methods of delivery of nucleic acid to T-cells and compositions for delivery thereof.

### BACKGROUND

T cells are part of the body's natural defense against disease and thus the ability to modify and reprogram them presents an attractive modality to target a variety of disease conditions. For example, a new therapy, referred to as “CAR T” is a promising approved treatment option for cancer, including certain hematological malignancies, such as relapsed acute B cell leukemia, aggressive B cell lymphoma and treatment-refractory multiple myeloma. CAR-T therapy relies on transfecting T cells, typically ex vivo, to produce what are known as “CAR-T cells”. The CAR-T cells express chimeric antigen receptor (CAR) on their surface that targets them in vivo to receptors on target cells of interest (Atsavapranee et al., 2021, EBioMedicine, 67:103354). In the case of cancer, this includes a tumour-specific antigen expressed on a population of cancerous cells. Upon binding to the surface antigen, the CAR T cells become activated and exert a desired therapeutic and/or prophylactic immune response against the target cell. While CAR T cell therapy is approved for cancer, it is also being investigated for cardiac fibrosis or to treat autoimmune diseases (e.g., to reduce immune responses after transplants).

Current approaches used to deliver nucleic acid coding for CAR are reliant on viral vectors. However, viral vectors are limited by the amount of genetic material that they can carry and there are safety concerns associated with permanent CAR expression (Billingsley et al., 2020, Nano Lett, 20(3):1578-1589). Electroporation is another method to deliver nucleic acid encoding for CAR to T cells. This involves introducing high voltage to make the cell membrane permeable for entry of the nucleic acid. However, the method is prone to cell damage requires the use of specialized equipment.

LNPs have been suggested as next-generation mRNA-based CAR T cell engineering. Most LNPs clinically approved for nucleic acid delivery are based on a formulation known as Onpattro™. The Onpattro™ formulation is a lipid nanoparticle-based short interfering RNA (siRNA) drug formulation for the treatment of polyneuropathies induced by hereditary transthyretin amyloidosis. The success of this LNP delivery system paved the way for the clinical development of the leading LNP-based COVID-19 mRNA vaccines.

The Onpattro™ LNP formulation consists of four main lipid components, namely: ionizable amino lipid, distearoylphosphatidylcholine (DSPC), cholesterol, and polyethylene glycol conjugated lipids (PEG-lipids) at respective molar amounts of 50/10/38.5/1.5. Onpattro™ is still considered the gold standard for comparison in studies of LNP-mediated efficacy and current approaches to LNP design make few deviations from the four-component system.

While strides have been made in research relating to LNP-mediated nucleic acid delivery, it is widely known that the Onpattro™ formulation largely accumulates in liver (hepatic) tissues. However, most T cells reside in the lymphoid tissues, such as the lymph nodes, thymus, blood, spleen and bone marrow. To target T cells, the ability of LNPs to be delivered in organs and tissues beyond the liver would greatly expand the clinical utility of T cell therapeutics. In order to improve the delivery of nucleic acid cargo to T cell populations, the particles should exhibit enhanced circulation lifetimes. Traditional approaches to achieve this rely on optimizing the levels of PEG-lipid in the LNP, but the inclusion of PEG-lipids in LNPs often results in transfection potencies that are low or results in unfavorable immune responses. While serum stable, large unilamellar vesicles (LUV) used for small molecule delivery have been prepared without PEG (Semple et al., 1996, Biochemistry, 35:2521-2525), its inclusion in solid-core type LNPs containing nucleic acid cargo is considered essential as it is thought to prevent aggregation of the particles during the formulation process (Kulkarni et al., 2020, Nanoscale, 12:23959-23966).

Kitte et al., 2023, Molecular Therapy, Methods & Clinical Development, 31:101139 observed that LNPs significantly prolonged cell efficacy in vitro as a result of extended CAR-mRNA and CAR T cell persistence. However, a lower number of CAR molecules per T cell were observed on CAR T cells transfected with LNPs relative to those produced by electroporation.

There is a need in the art to improve the ex vivo or in vivo delivery of nucleic acid encoding nucleic acid to modify a T cell, such as nucleic acid encoding CAR.

### SUMMARY

The present disclosure addresses one or more of the foregoing problems in the prior art and/or provides useful alternatives to known compositions for the delivery of cargo to T cells to thereby produce modified T cells to treat, prevent or ameliorate a disease or condition.

The present disclosure is based on the finding that an LNP formulation (herein “lcLNP™”) that includes elevated levels of neutral lipid, such as greater than 20 mol %, exhibits surprising improvements in the delivery of nucleic acid to T cells relative to more conventional LNP formulations, such as an Onpattro™ LNP (hereinafter referred to as a “baseline LNP”, “baseline formulation” or “baseline”) having 10 mol % DSPC. In particular, populations of T cells transfected with LNPs described herein exhibit a higher percentage of modified cells (e.g., expressing a protein encoded by mRNA) relative to T cell populations transfected with the baseline LNP. In another embodiment, T cells transfected with LNPs with editing cargo described herein exhibit a higher percentage of gene editing than the baseline LNP. This latter observation was most pronounced when LNPs were prepared without PEG-lipid.

In some embodiments, the LNPs herein may be used to deliver nucleic acid encoding for the chimeric antigen receptor to a variety of T cell sub-types, in turn producing CAR T cells to treat or prevent a wide range of diseases or conditions. Alternatively, or additionally, LNPs herein could be used to generate allogeneic T cells by knock-out of T cell receptors that cause immunogenicity in a subject.

According to one aspect of the disclosure, there is provided a method for delivery of nucleic acid to a T cell to produce a modified T cell, the method comprising contacting a lipid nanoparticle encapsulating the nucleic acid with the T cell ex vivo or in vivo, thereby causing cellular uptake of the nucleic acid, the lipid nanoparticle having between 30 mol % and 70 mol % of a neutral or zwitterionic amphipathic lipid having a net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid modifies the T cell by: (i) altering expression of a protein, polypeptide or peptide in the T-cell; and/or (ii) expressing an endogenous or exogenous protein, polypeptide or peptide in the T cell, thereby producing the modified T cell, wherein the modified T cell thereby produced provides a therapeutic, prophylactic or ameliorative effect in vivo.

According to one embodiment of the foregoing aspect, the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

According to another example of the foregoing aspect or embodiment, the contacting is in vivo and the T cell is in the blood, spleen or bone marrow of a subject.

In a further example of the foregoing aspect or any embodiment thereof, the nucleic acid is mRNA for expressing an endogenous or exogenous protein, polypeptide or peptide in the T-cell.

According to a further example of the foregoing aspect or any embodiment thereof, the endogenous or exogenous protein, polypeptide or peptide is a chimeric antigen receptor.

In a further example of the foregoing aspect or any embodiment thereof, the nucleic acid is part of a nucleic acid editor.

According to a further example of the foregoing aspect or any embodiment thereof, the nucleic acid editor is a Cas-based editor, transcription activator-like effector nuclease (TALEN), megaTAL, zinc finger nuclease (ZFN), Adenosine Deaminase Acting on RNA (ADAR), prime editor, base editor, epigenetic editor, transposase, meganuclease, ARCUS gene editing system or a combination thereof.

According to a further example of the foregoing aspect or any embodiment thereof, the Cas-based editor is CRISPR and comprises a guide RNA and an mRNA or vector DNA coding for a Cas nuclease.

In a further example of the foregoing aspect or any embodiment thereof, the lipid nanoparticle is used to treat a disease or disorder that is an immunological disease or disorder.

According to a further example of the foregoing aspect or any embodiment thereof, the lipid nanoparticle is used to treat a disease or disorder that is a cancer.

In another example of the foregoing aspect or any embodiment thereof, the cancer is a haematological cancer.

According to a further aspect of the disclosure, there is provided a lipid nanoparticle comprising an encapsulated nucleic acid for ex vivo or in vivo delivery to a T cell to produce a modified T cell having a therapeutic, prophylactic or ameliorative effect in vivo, the lipid nanoparticle having between 30 mol % and 70 mol % of neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid is for modifying the T cell by: (i) altering expression of an endogenous protein, polypeptide or peptide in the T-cell; and/or (ii) expressing an endogenous or exogenous protein, polypeptide or peptide in the T cell, optionally wherein, the lipid nanoparticle, when encapsulating eGFP, exhibits at least a 10% increase in expression of eGFP in CD4 or CD8 T cells of the blood or bone marrow relative to a baseline formulation having 50/10/38.5/1.5 mol/mol ionizable cationic lipid, DSPC, cholesterol and PEG-lipid measured at 24 hours post-injection in a mouse model.

According to a further aspect of the disclosure, there is provided a lipid nanoparticle comprising an encapsulated nucleic acid for ex vivo or in vivo delivery to a T cell to produce a modified T cell having a therapeutic, prophylactic or ameliorative effect in vivo, the lipid nanoparticle having between 30 mol % and 70 mol % of neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid is for modifying the T cell and wherein the lipid nanoparticle has less than 1.5 mol % PEG-lipid.

According to some embodiments of either one of the foregoing aspects, the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

According to a further example of either one of the foregoing aspects or any embodiment thereof, the lipid nanoparticle is for modifying the T cell in vivo in a subject's blood, spleen or bone marrow.

According to a further example of either one of the foregoing aspects or any embodiment thereof, the nucleic acid is for expressing an endogenous or exogenous protein or peptide in the T-cell.

According to a further example of either one of the foregoing aspects or any embodiment thereof, the endogenous or exogenous protein or peptide is a chimeric antigen receptor.

According to a further example of either one of the foregoing aspects or any embodiment thereof, the nucleic acid is part of a nucleic acid editor.

According to a further example of either one of the foregoing aspects or any embodiment thereof, the nucleic acid editor is a Cas-based editor, transcription activator-like effector nuclease (TALEN), megaTAL, zinc finger nuclease (ZFN), Adenosine Deaminase Acting on RNA (ADAR), prime editor, base editor, epigenetic editor, transposase, meganuclease, ARCUS gene editing system or a combination thereof.

According to a further example of either one of the foregoing aspects or any embodiment thereof, the Cas-based editor is CRISPR and comprises a guide RNA and an mRNA or vector DNA coding for a Cas nuclease.

According to a further example of either one of the foregoing aspects or any embodiment thereof, the lipid nanoparticle is for use to produce the modified T cell having the therapeutic, prophylactic or ameliorative effect in vivo.

According to a further example of either one of the foregoing aspects or any embodiment thereof, the lipid nanoparticle is used to treat a disease or disorder that is a cancer.

According to a further example of either one of the foregoing aspects or any embodiment thereof, the lipid nanoparticle is used to treat a cancer that is a haematological cancer.

According to a further example of either one of the foregoing aspects or any embodiment thereof, the lipid nanoparticle is used to treat a disease or disorder that is an immunological disease or disorder.

According to a further aspect of the disclosure, there is provided an ex vivo CD4 or CD8 T cell preparation comprising the lipid nanoparticle as described in any one of the foregoing aspects or embodiments thereof.

### BRIEF DESCRIPTION OF THE DRAWINGS

FIG. 1A shows flow cytometry data for untreated and LNP-treated primary human CD8+ T cells staining for dead cells at 48 hours post treatment. The LNPs examined encapsulate mRNA encoding for eGFP and contained 10 mol % DSPC (nMC3:DSPC:Chol:PEG₂₀₀₀-DMG-lipid (50:10:38.5:1.5 mol:mol) (Baseline)); or 50 mol % DSPC (ionizable cationic lipid:DSPC:Chol (28.15:50:21.85 mol:mol) (“lcLNP™-PEG-less) and were titrated at doses of 1, 2, 4, 6 and 8 μg. The ionizable cationic lipids used in the lcLNP™ compositions were 1 and 2 as described in Table 1 of Example 1.

FIG. 1B shows flow cytometry data for untreated and LNP-treated primary human CD8+ T cells quantifying for green fluorescent protein (GFP) expression at 48 hours post treatment. The LNP formulations are the baseline and lcLNP™ 50 mol % PEG-less formulations described above and set forth in Table 1 of Example 1.

FIG. 2A and FIG. 2B show ex vivo representative flow cytometry gating strategy for TCR/CD3 for the flow cytometry data of FIGS. 3 and 4 below.

FIG. 3A is a gel showing genomic DNA undigested and digested with T7 endonuclease (T7EI) after treatment of CD8+ T cells ex vivo with LNP formulations encapsulating mRNA encoding Cas9 and T cell receptor α constant (TRAC) gRNA or scrambled gRNA at a dose of 2.5 μg. The LNPs examined were baseline IL1, composed of ionizable lipid 1 (IL1):DSPC:Chol:PEG₂₀₀₀-DMG (50:10:38.5:1.5 mol:mol); baseline IL2, ionizable lipid 2 (IL2):DSPC:Chol:PEG₂₀₀₀-DMG (50:10:38.5:1.5 mol:mol); lcLNP™ PEG-less IL2, IL2:DSPC:Chol (28.15:50:21.85 mol:mol); and lcLNP™ PEG-less IL2, with scrambled gRNA composed of IL2:DSPC:Chol (28.15:50:21.85 mol:mol). The ionizable cationic lipids used in the lcLNP™ compositions were lipids 1 and 2 as described in Table 3 of Example 2.

FIG. 3B is a graph showing ex vivo efficiency (%) INDEL and knockout score for CD8+ T cells treated with the LNPs set out in FIG. 3A as well as untreated samples using Interference of CRISPR Editing (ICE) at 48 hours post-LNP treatment to the T cells.

FIG. 3C is a graph showing ex vivo flow cytometry T cell receptor (TCR) data for CD8+ T cells treated with the LNPs set out in FIG. 3A as well as untreated samples at 8 days post-LNP treatment.

FIG. 3D is a graph showing quantification of TCR and CD3 expression ex vivo normalized to un-transfected cells by flow cytometry for CD8+ T cells treated with the LNPs set out in FIG. 3A as well as untreated samples at 8 days post-LNP treatment.

FIG. 4A shows ex vivo T cell receptor (TCR) expression by flow cytometry 8 days post LNP treatment in primary human CD4+ and CD8+ cells treated with PEG-less lcLNP™ having ionizable lipid 2:DSPC:Chol (28.15:50:21.85 mol:mol) encapsulating Cas9 and TRAC gRNA. Ionizable lipid 2 is set forth in Table 3 of Example 2.

FIG. 4B shows quantification of TCR and CD3 expression normalized to un-transfected samples by the flow cytometry comparing CD4+ and CD8+ T cells treated with PEG-less lcLNP™ having ionizable lipid 2 (IL2):DSPC:Chol (28.15:50:21.85 mol:mol) encapsulating Cas9 and TRAC gRNA. Ionizable lipid 2 is set forth in Table 3 of Example 2.

FIG. 5A shows size (nm), polydispersity (PDI) and encapsulation % of lipid nanoparticles composed of 50:10:38.5:1.5 mol:mol ionizable cationic lipid 1:DSPC:cholesterol:PEG₂₀₀₀-DMG (baseline) and lcLNP™ composed of 27.4:50:21.1:1.5 ionizable cationic lipid:DSPC:Chol:PEG₂₀₀₀-DMG. The ionizable cationic lipids used in the lcLNP™ compositions were lipids 1 (nMC3 control), 2, 3, 4 and 5 as described in Table 5 of Example 4.

FIG. 5B shows the percentage of enhanced green fluorescent protein (eGFP) positive T cells (% eGFP) in bone marrow as indicated 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 3, 4 and 5 as described in Table 5 of Example 4.

FIG. 5C shows the percentage of enhanced green fluorescent protein (eGFP) positive CD4⁺ and CD8⁺ T cells (% eGFP) in bone marrow 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 3, 4 and 5 as described in Table 5 of Example 4.

FIG. 5D shows the percentage of enhanced green fluorescent protein (eGFP) positive cells (% eGFP) in blood T cells as indicated 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 3, 4 and 5 as described in Table 5 of Example 4.

FIG. 5E shows the percentage of enhanced green fluorescent protein (eGFP) positive CD4⁺ and CD8⁺ T cells (% eGFP) in blood indicated 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 3, 4 and 5 as described in Table 5 of Example 4.

FIG. 6A shows size (nm), polydispersity (PDI) and encapsulation % of lipid nanoparticles composed of 50:10:38.5:1.5 mol/mol ionizable cationic lipid 1:DSPC:cholesterol:PEG₂₀₀₀-DMG (baseline) and lcLNP™ composed of 27.4:50:21.1:1.5 ionizable cationic lipid:DSPC:Chol:PEG₂₀₀₀-DMG (mol/mol). The ionizable cationic lipids used in the lcLNP™ compositions were 1 (control), 2, 6 and 7 as described in Table 5 of Example 4.

FIG. 6B shows the percentage of enhanced green fluorescent protein (eGFP) positive cells (% eGFP) in bone marrow indicated 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 6 and 7 as described in Table 5 of Example 4.

FIG. 6C shows the percentage of enhanced green fluorescent protein (eGFP) positive CD4⁺ and CD8⁺ T cells (% eGFP) in bone marrow as indicated 24 hours post-intravenous injection of eGFP-mRNA LNPs having 10 mol % DSPC (nMC3:DSPC:Chol:PEG₂₀₀₀-DMG-lipid (50:10:38.5:1.5) (baseline)); or 50 mol % DSPC (ionizable cationic lipid:DSPC:Chol:PEG-lipid (27.4:50:21.1:1.5 mol:mol) (“lcLNP™”)) into C57Bl/6 mice. The ionizable cationic lipids used in the lcLNP™ compositions were 1 (control), 2, 6 and 7 as described in Table 5 of Example 4.

FIG. 7A depicts the homology directed repair (HDR) template comprising a sequence encoding eGFP flanked by left homology arm (LHA) and right homology arm (RHA) sequences.

FIG. 7B shows flow cytometry data for un-transfected primary human CD4+ T cells in the ex vivo knock-out/knock-in study set forth in Example 5.

FIG. 7C shows flow cytometry data for primary human CD4+ T cells treated with lcLNP™ with Cas9 mRNA:TCR gRNA (1:1 wt:wt) in the ex vivo knock-out/knock-in study set forth in Example 5.

FIG. 7D shows flow cytometry data for primary human CD4+ T cells treated with lcLNP™ having Cas9 mRNA:TCR gRNA:GFP homology directed repair (HDR) (1:1:3 wt:wt) DNA in the ex vivo knock-out/knock-in study set forth in Example 5.

FIG. 7E shows flow cytometry data for primary human CD4+ T cells treated with lcLNP™ having Cas9 mRNA:TCR gRNA:GFP HDR DNA (1:1:3 wt:wt) in the ex vivo knock-out/knock-in study with inhibitor set forth in Example 5.

### DETAILED DESCRIPTION

### Cargo

As used herein, the term “encapsulation,” with reference to incorporating the nucleic acid cargo within a lipid nanoparticle refers to any association of the nucleic acid with any lipid component or compartment of the lipid nanoparticle. In one example of the disclosure the nucleic acid is present in the core of the LNP.

The LNPs herein comprise a nucleic acid that modifies a T cell ex vivo or in vivo by (i) expressing an endogenous or exogenous protein, polypeptide or peptide in the T cell, and/or (ii) altering expression of a protein, polypeptide or peptide in the T-cell, thereby producing the modified T cell. Examples of each are described below.

By the term “endogenous” with reference to a protein, polypeptide or peptide in the T cell, it is meant a protein, polypeptide or peptide, or fragment thereof, that is encoded by the genetic material of an un-modified T cell.

By the term “exogenous” with reference to a protein, polypeptide or peptide in the T cell, it is meant a protein, polypeptide or peptide, or fragment thereof, that is not encoded by the genetic material of an un-modified T cell.

By the term “modified T cell”, it is meant a T cell that is modified by introduction of a nucleic acid delivered by the LNPs described herein, which nucleic acid alters an expression profile of the cell.

The nucleic acid that modifies the T cell includes, without limitation, RNA, including small interfering RNA (siRNA), small nuclear RNA (snRNA), small nucleolar RNA (snoRNA), micro RNA (miRNA), guide RNA (gRNA), including single guide RNA (sgRNA), prime editing guide RNA (pegRNA), messenger RNA (mRNA), small activating RNA (saRNA), self-replicating RNA (srRNA), transamplifying RNA (taRNA), circular RNA (circRNA), long noncoding RNA (lncRNA), and transfer RNA (tRNA); and DNA such as vector DNA and linear DNA, or hybrids thereof. The nucleic acid length can vary and can include nucleic acid of 1-50,000 nucleotides in length. The nucleic acid can be in any form, including single stranded DNA or RNA, double stranded DNA or RNA, or hybrids thereof. Single stranded nucleic acid includes antisense oligonucleotides. The nucleic acid may be conjugated to another molecule, including a targeting moiety. An example of such a nucleic acid conjugate is an antibody-nucleic acid conjugate, or an oligosaccharide-nucleic acid conjugate, such as a GalNAc-nucleic acid conjugate.

### Expressing an Endogenous or Exogenous Protein, Polypeptide or Peptide

Expressing an endogenous or exogenous protein, polypeptide or peptide in a T cell in some embodiments includes formulating the lipid nanoparticle with a nucleic acid that encodes for or causes expression of the protein, polypeptide or peptide in a T cell.

The nucleic acid cargo that encodes for the protein, polypeptide or peptide comprises a nucleotide sequence that, when expressed, produces the protein, polypeptide or peptide in the T cell. Without limitation, this includes a protein, polypeptide comprising an antigen for expression on the surface of the T cell. A nucleic acid that causes expression of the protein, polypeptide or peptide in the T cell includes a nucleic acid that encodes a protein, polypeptide or peptide that regulates the expression of another nucleic acid molecule within the T-cell. Examples include regulatory elements that encode for protein, polypeptide or a peptide for upregulation of a targeted nucleic acid in the T cell. In another embodiment, the expressed protein, polypeptide or peptide is part of an editing complex. In another embodiment, the protein, polypeptide or peptide is expressed from a cassette or transgene (delivered via the LNP as a cargo) that is inserted into genetic material of the T cell, (e.g., such as a “knock-in” of a TRAC locus).

As used herein, the term “messenger RNA” or “mRNA”, refers to a polynucleotide that encodes and expresses at least one protein, polypeptide or peptide. The term is meant to include mRNA that is circular or linear as well as small activating RNA (saRNA) and trans-amplifying RNA (taRNA).

The concentration of mRNA in the LNP may be between 0.01 and 20 mg/mL or between 0.01 and 10 mg/mL or between 0.05 and 5 mg/mL or between 0.075 and 4 mg/mL.

The mRNA, as used herein, encompasses both modified and unmodified mRNA. In one embodiment, the mRNA comprises one or more coding and non-coding regions. The mRNA can be purified from natural sources, produced using recombinant expression systems and optionally purified, or may be chemically synthesized.

In those embodiments in which an mRNA is chemically synthesized, the mRNA can comprise nucleoside analogs such as analogs having chemically modified bases or sugars, and/or backbone modifications. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The mRNAs of the disclosure may be synthesized according to any of a variety of known methods. For example, mRNAs in certain embodiments may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor.

In some embodiments, in vitro synthesized mRNA may be purified before encapsulation to remove undesirable impurities including various enzymes and other reagents used during mRNA synthesis.

The present disclosure may be used to formulate mRNAs of a variety of lengths. In some embodiments, the present disclosure may be used to formulate and encapsulate in vitro synthesized mRNA ranging from about 1-20 kb, about 1-20 kb, about 1-15 kb, about 1-10 kb, about 2-20 kb, about 2-15 kb, about 2-10 kb, about 3-20 kb, about 3-15 kb, about 3-10 kb, about 3-7 kb, about 5-20 kb, about 5-15 kb, about 5-12 kb, about 5-10 kb, about 8-20 kb, or about 8-15 kb in length.

In those embodiments in which the mRNA is linear, the synthesis includes the addition of a “cap” on the 5′ end, and a “tail” on the 3′ end. The presence of the cap provides resistance to nucleases found in most eukaryotic cells. The presence of a “tail” serves to protect the mRNA from exonuclease degradation.

In some embodiments, mRNAs include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In a further embodiment, the mRNA is circular. Advantageously, such mRNA lacks 5′ and 3′ ends and thus may be more stable in vivo due to its resistance to degradation by exonucleases. The circular mRNA may be prepared by any known method, including any one of the methods described in Deviatkin et al., 2023, “Cap-Independent Circular mRNA Translation Efficiency”, Vaccines, 11(2), 238, which is incorporated herein by reference. Translation of the circular mRNA is carried out by a cap-independent initiation mechanism.

While mRNA provided from in vitro transcription reactions may be desirable in certain embodiments, other sources of mRNA are contemplated, such as mRNA produced from bacteria, fungi, plants, and/or animals.

The mRNA sequence may comprise a reporter gene sequence, although the inclusion of a reporter gene sequence in pharmaceutical formulations for administration is optional. Such sequences are incorporated into mRNA for in vivo studies in animal models to assess biodistribution.

In another embodiment, the cargo is a DNA vector. The encapsulated DNA vector may be administered to a subject for the purpose of repairing, enhancing or blocking or reducing the expression of a cellular protein or peptide.

As will be appreciated by those of skill in the art, the vectors may encode promoter regions, operator regions or structural regions. The DNA vectors may contain double-stranded DNA or may be composed of a DNA-RNA hybrid. Non-limiting examples of double-stranded DNA include structural genes, genes including operator control and termination regions, and self-replicating systems such as vector DNA.

Single-stranded nucleic acids include antisense oligonucleotides (complementary to DNA and RNA), ribozymes and triplex-forming oligonucleotides. In order to have prolonged activity, the single-stranded nucleic acids will most advantageously have some or all of the nucleotide linkages substituted with stable, non-phosphodiester linkages, including, for example, phosphorothioate, phosphorodithioate, phophoroselenate, or O-alkyl phosphotriester linkages.

The DNA vectors may include nucleic acids in which modifications have been made in one or more sugar moieties and/or in one or more of the pyrimidine or purine bases. Such sugar modifications may include replacement of one or more hydroxyl groups with halogens, alkyl groups, amines, azido groups or functionalized as ethers or esters. In another embodiment, the entire sugar may be replaced with sterically and electronically similar structures, including aza-sugars and carbocyclic sugar analogs. Modifications in the purine or pyrimidine base moiety include, for example, alkylated purines and pyrimidines, acylated purines or pyrimidines, or other heterocyclic substitutes known to those of skill in the art.

The DNA vector may be modified in certain embodiments with a modifier molecule such as a peptide, protein, steroid or sugar moiety. Modification of a DNA vector with such molecule may facilitate delivery to a target site of interest. In some embodiments, such modification translocates the DNA vector across a nucleus of a target cell. By way of example, a modifier may be able to bind to a specific part of the DNA vector (typically not encoding of the gene-of-interest), but also has a peptide or other modifier that has nucleus-homing effects, such as a nuclear localization signal. A non-limiting example of a modifier is a steroid-peptide nucleic acid conjugate as described by Rebuffat et al., 2002, Faseb J. 16(11):1426-8, which is incorporated herein by reference. The DNA vector may contain sequences encoding different proteins or peptides. Promoter, enhancer, stress or chemically-regulated promoters, antibiotic-sensitive or nutrient-sensitive regions, as well as therapeutic protein encoding sequences, may be included as required. Non-encoding sequences may be present as well in the DNA vector.

The nucleic acids used in the present disclosure can be isolated from natural sources, obtained from such sources as ATCC or GenBank libraries or prepared by synthetic methods. Synthetic nucleic acids can be prepared by a variety of solution or solid phase methods. Generally, solid phase synthesis is preferred. Known procedures for solid phase synthesis of nucleic acids by phosphite-triester, phosphotriester, and H-phosphonate chemistries are widely available.

In one embodiment, the DNA vector is double stranded DNA and comprises more than 700 base pairs, more than 800 base pairs or more than 900 base pairs or more than 1000 base pairs.

### Altering Expression of a Protein, Polypeptide or Peptide in the T-Cell

In another embodiment of the disclosure, the nucleic acid alters expression of a protein, polypeptide or peptide in the T-cell. In some embodiments, the nucleic acid reduces expression of a protein, polypeptide or peptide in the T-cell.

For example, the cargo may be an siRNA that reduces expression of a gene. An siRNA becomes incorporated into endogenous cellular machineries to result in mRNA breakdown, thereby preventing transcription.

The siRNA encompassed by embodiments of the disclosure may be used to specifically inhibit expression of a wide variety of target polynucleotides in a T cell. The siRNA molecules targeting specific polynucleotides for any therapeutic, prophylactic or diagnostic application may be readily prepared according to procedures known in the art. An siRNA target site in a T cell may be selected and corresponding siRNAs may be chemically synthesized, created by in vitro transcription, or expressed from a vector or PCR product. The siRNA may be double-stranded RNA, or a hybrid molecule comprising both RNA and DNA, e.g., one RNA strand and one DNA strand. The siRNA may be of a variety of lengths, such as 1 to 30 nucleotides in length or 15 to 30 nucleotides in length or 20 to 25 nucleotides in length. In certain embodiments, the siRNA is double-stranded and has 3′ overhangs or 5′ overhangs. In certain embodiments, the overhangs are UU or dTdT 3′. In particular embodiments, the siRNA comprises a stem loop structure.

In a further embodiment, the cargo molecule is a microRNA or small nuclear RNA. Micro RNAs (miRNAs) are short, noncoding RNA molecules that are transcribed from genomic DNA, but are not translated into protein. These RNA molecules are believed to play a role in regulation of gene expression by binding to regions of target mRNA. Binding of miRNA to target mRNA may downregulate gene expression, such as by inducing translational repression, deadenylation or degradation of target mRNA. Small nuclear RNA (snRNA) are typically longer noncoding RNA molecules that are involved in gene splicing. The snRNA molecules may have therapeutic or diagnostic importance in diseases that are an outcome of splicing defects.

In one embodiment, the LNP-encapsulated cargo edits a T cell to produce a desired modification to treat, prevent or ameliorate a disease or condition. A non-limiting example includes editing a T cell to improve CAR T cell therapy. For example, gene editing of a T cell may be used to ameliorate CAR T cell dysfunction (e.g., T cell exhaustion), modulate cytokine production or knocking in nucleic acid, such as CAR cassettes at specific genomic locations. In another embodiment, editing cargo can be used to produce allogeneic CAR T cells. (See Dimitri et al., 2022, Molecular Cancer, 21, article No. 78, which is incorporated herein by reference).

### Editing Cargo

As used herein, the term “editing cargo” includes a protein and/or nucleic acid-based system that causes modification of a T cell at a specific locus or loci to produce a desired modification to treat, prevent or ameliorate a disease or condition.

As used herein, the term “nucleic acid editor” includes a protein and/or nucleic acid-based system that causes modification of any nucleic acid of a T cell at a specific locus or loci to produce a desired modification to treat, prevent or ameliorate a disease or condition.

The cargo may comprise a nucleic acid that encodes for a protein or peptide that forms part of a nucleic acid editing complex. A “nucleic acid editing complex” includes without limitation protein and/or nucleic acid-based systems in which nucleic acid is inserted, deleted, modified (e.g., epigenetic editing) or replaced in the genetic material of an organism at a site-specific location.

The nucleic acid editing complex may be used for ex vivo or in vivo genetic modification of a T cell and includes post-translational modifications.

Alternatively or additionally, the cargo comprises a peptide or protein that is part of an editor or forms an editing complex.

The nucleic acid editing complex includes, without limitation, Cas-based (e.g., CRISPR or non-CRISPR), transcription activator-like effector nuclease (TALEN), megaTALs, zinc finger nuclease (ZFN), Adenosine Deaminase Acting on RNA (ADAR), prime editors, base editors, epigenetic, transposase, meganuclease, ARCUS gene editing systems or any variant or combination thereof. These nucleic acid editing systems are exemplary and include any cargo that can modify genetic material (including RNA transcripts and non-coding regions) of a cell to treat, prevent or ameliorate a disorder or disease. Without limitation, the gene editing system may include those that are designed by a process referred to as Directed Nuclease Editor (DNE), which is known to those of skill in the art.

Cas-based editing systems comprise CRISPR and non-CRISPR gene editing systems. In addition, the editing systems include those that cut DNA as well as epigenetic editing systems that modify nucleic acid markers, as discussed below.

The CRISPR gene editing cargo most advantageously comprises nucleic acid (e.g., mRNA) encoding for one or more of a Class II Cas nuclease family of proteins and a guide RNA. The nucleases encoded by the nucleic acid are enzymes with DNA endonuclease activity and can be directed to cleave a desired nucleic acid target by an appropriate guide RNA. The nuclease and guide RNA form a complex referred to as a ribonucleoprotein (RNP). In some embodiments, the nuclease is a Class II CRISPR enzyme, which is further subdivided into Types II, V and VI. According to one embodiment, the mRNA encodes for a Cas protein that is part of a Type II CRISPR/Cas system, such as a Cas9 protein or a Cpf1 protein.

In another embodiment, the mRNA encodes for a Cas protein that is part of a Type V CRISPR/Cas system, such as Cas12a. In another embodiment, the mRNA encodes for a Cas protein that is a Cas 13a, which is an RNA endonuclease and cleaves single-stranded RNA.

The guide RNA can direct the Cas nuclease to the target sequence on a target nucleic acid molecule, where the guide RNA hybridizes to the target sequence and the Cas nuclease cleaves or modulates the sequence. In some embodiments, the guide RNA binds to a class 2 nuclease, thereby providing specificity of cleavage.

Guide RNAs for the CRISPR/Cas9 nuclease system include CRISPR RNA (crRNA) or tracr RNA (tracr). In some embodiments, the crRNA can include a targeting sequence that is complementary to and hybridizes to a target sequence on a target nucleic acid molecule. The crRNA can also include a flagpole that is complementary to, and hybridize to, a portion of tracrRNA. In some embodiments, the crRNA can correspond to the structure of a naturally-occurring crRNA transcribed from a bacterial CRISPR locus, wherein the targeting sequence acts as a spacer for the CRISPR/Cas9 system. The flagpole corresponds to the part of the repetitive sequence adjacent to the spacer above the CRISPR locus.

The guide RNA of the RNP can target any sequence of interest through the targeting sequence of crRNA. In some embodiments, the targeting sequence of the guide RNA and the target sequence on the target nucleic acid molecule may be 100% complementary. In other embodiments, the targeting sequence of the guide RNA and the target sequence on the target nucleic acid molecule can comprise at least one mismatch.

The length of the targeting sequence may depend on the RNP system and components used. For example, different Cas proteins from different bacterial species have various optimal targeting sequence lengths. Thus, the targeting sequences are: 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length can be included. In some embodiments, the targeting sequence can comprise a length of 18 to 24 nucleotides. In some embodiments, the targeting sequence can comprise 19-21 nucleotides in length. In some embodiments, the targeting sequence can comprise a length of 20 nucleotides.

In some embodiments, the editing system includes Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csbl11, Csb2, Csb3, Csx17, Csx14, Csx10, Csx16, CsaX, Csx3, Csx1, Csx15, Csf1, Csf2, Csf3, Csf4, homologs thereof, or modified versions thereof.

As noted, non-CRISPR, Cas-based gene editing systems are encompassed by embodiments of the disclosure as well. A Cas-based editing system may include a Cas enzyme fused to deaminase (Luo et al., 2020, Microbial Cell Factories, 19(93), incorporated herein by reference). An example is a cytosine base editor or an adenine base editor produced by fusing endonuclease Cas to cytosine deaminase pmCDA1 or heterodimer adenine deaminase TadA-TadA. A further non-limiting example is Cas fused to reverse transcriptase (Mohr et al., 2018, Mol Cell., 72(4):700-714, incorporated herein by reference).

Fanzor is a eukaryotic RNA-guided endonuclease that could function as a gene editor. (See Saito et al., 2023, Nature 620:660-668, which is incorporated herein by reference). In some embodiments, Fanzor proteins use RNA as a guide to target DNA precisely and can be modified to edit a T cell using LNPs described herein. In some examples, the compact Fanzor systems may have the ability to facilitate more improved delivery than CRISPR-Cas systems.

In those embodiments in which the cargo is a TALEN, the cargo comprises a nucleic acid encoding a peptide having a Transcription activator-like (TAL) effector DNA binding domain, a fragment or a variant thereof. In an embodiment, the system comprises a nucleic acid encoding a peptide having nuclease activity, e.g., endonuclease activity. In an embodiment, the peptide having nuclease activity is a type-II restriction 1-like endonuclease, e.g., a Fokl endonuclease.

In those embodiments in which the cargo is a ZFN, the nucleic acid may encode a peptide having: a Zinc finger DNA binding domain, a fragment or a variant thereof, and/or nuclease activity, e.g., endonuclease activity. In an embodiment, the Zn finger binding domain comprises 1, 2, 3, 4, 5, 6, 7, 8 or more Zinc fingers. In an embodiment, the peptide having nuclease activity is a type-II restriction 1-like endonuclease, e.g., a Fokl endonuclease.

Adenosine Deaminase Acting on RNA (ADAR) is another editing cargo encompassed by embodiments of the disclosure that may be used for post-transcriptional modification of RNA. Examples include ADAR1 and ADAR2. ADAR1 may catalyze posttranscriptional deamination of C6 of adenosines in dsRNA, converting them to inosines (see Song et al., 2022, PMC, 13(1):e1665, incorporated herein by reference).

Meganucleases are enzymes in the endonuclease family that may induce homologous recombination, generate mutations and alter reading frames. The meganuclease includes homing endonucleases that are intron or intein endonucleases. In one embodiment, the meganuclease is from the LAGLIDADG family, a GIY-YIG endonuclease, an HNH endonuclease, a His-Cys box endonuclease or a PD-(D/E)XK endonuclease. Meganucleases may be combined with components of other gene editing system. In one embodiment, a DNA binding domain from a transcription activator-like (TAL) effector is combined with a meganuclease to produce a “megaTAL”. In another embodiment, a meganuclease may be fused to a DNA end-processing enzyme to promote an error-prone non-homologous end joining.

ARCUS nuclease is a gene editing system based on I-CreI, which is a kind of homing endonuclease that evolved in the algae Chlamydomonas reinhardtii. In some embodiments, the nuclease can deactivate itself after gene editing, thereby reducing off-targeting. ARCUS nucleases in some embodiments can generate a unique cleavage site that is a four-base-pair, 3′ overhang and may be able to carry out gene insertion, gene excision, gene repair or a combination thereof.

Epigenetic editing is also encompassed by examples of the disclosure. Such editing of genetic material does not cut nucleic acid but rather alters epigenomic marks “adorning” DNA. Changing the epigenic signature of a T cell can serve to modify an epigenetic signature of the cell and change its transcriptional profile.

Further examples of effector proteins include DNA methyltransferase, a fragment (e.g., a biologically active fragment) or variant thereof (e.g, DNMT1, DNMT2 DNMT3A, DNMT3B, DNMT3L, or CpG methyltransferase (M. Sssl)); or a poly comb repressive complex or a component thereof, e.g, PRC1 or PRC2, or PR-DUB, or a fragment (e.g, biologically active fragment) or a variant thereof.

In an embodiment, the epigenetic editor comprises a molecule that modifies chromatin architecture and/or modifies a histone. In an embodiment, the epigenetic modulator is a molecule that modifies chromatin architecture, e.g., a SWI/SNF remodeling complex or a component thereof. In an embodiment, the epigenetic modulator is a molecule that modifies a histone, e.g., methylates and/or acetylates a histone, e.g., a histone modifying enzyme or a fragment (e.g., biologically active fragment) or a variant thereof, e.g., HMT, HDM, HAT, or HD AC.

### Structural Non-Cationic Lipid

The LNP generally includes one or more structural lipids, meaning an amphipathic lipid that allows for the formation of particles and generally bears no net charge at physiological pH (7.4). The term includes neutral as well as zwitterionic lipids that impart substantially no charge at physiological pH to the LNP and includes phospholipids. In alternative embodiments, the structural lipid is a non-cationic lipid.

As used herein “substantially no charge”, means a net surface charge of about zero, or near neutral at physiological pH, such as without limitation about −2.5 mV to about 2.5 mV, or −5 mV to about 5 mV

In some embodiments, the structural lipid is a phosphatidylcholine lipid (PC-lipid) selected from distearoylphosphatidylcholine (DSPC), dioleoylphosphatidylcholine (DOPC), dimyristoylphosphatidylcholine (DMPC), 1-palmitoyl-2-oleoyl-phosphatidylcholine (POPC) and dipalmitoyl-phosphatidylcholine (DPPC) and a sphingomyelin, include those with a phosphatidylcholine head group.

The structural, neutral, zwitterionic or non-cationic lipid content in some embodiments is greater than 20 mol %, greater than 25 mol %, greater than 30 mol %, greater than 32 mol %, greater than 34 mol %, greater than 36 mol %, greater than 38 mol %, greater than 40 mol %, greater than 42 mol %, greater than 44 mol %, greater than 46 mol %, greater than 48 mol % or greater than 50 mol %. In some embodiments, the upper limit of helper lipid content is 70 mol %, 65 mol %, 60 mol %, 55 mol %, 50 mol % or 45 mol %. The disclosure also encompasses sub-ranges of any combination of the foregoing numerical upper and lower limits.

For example, in certain embodiments, the phosphatidylcholine lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

In some embodiments, the phosphatidylcholine lipid content is primarily composed of DSPC or DMPC or primarily DSPC. In such embodiments, the mixture may have a DSPC content of at least 20, 30, 35, 40 or 45 mol % based on the total lipid content of the lipid nanoparticle with the balance of the phosphatidylcholine lipid content being another phosphatidylcholine lipid(s). In another embodiment, the phosphatidylcholine content is made up of at least 40 or 50 mol % DSPC relative to the total phosphatidylcholine content of the lipid nanoparticle.

In certain embodiments, the DSPC lipid content is from 20 mol % to 80 mol % or 25 mol % to 60 mol % or 30 mol % to 60 mol % or 35 mol % to 60 mol % or 40 mol % to 60 mol % or 42 mol % to 58 mol %, or 43 mol % to 57 mol % or 44 mol % to 56 mol % or 45 mol % to 55 mol % of total lipid present in the lipid nanoparticle.

The LNP may comprise additional lipids besides a neutral or structural lipid. For example, the LNP may comprise structural lipids that have a net positive or negative charge at physiological pH. Generally, as discussed below, such lipids may be present at less than 10 mol % or less than 5 mol %.

In alternative embodiments, the mixture may have a DMPC content of at least 20, 30, 35, 40 or 45 mol % based on the total lipid content of the lipid nanoparticle. In another embodiment, the phosphatidylcholine content is made up of at least 40 mol % DMPC relative to the total phosphatidylcholine content of the lipid nanoparticle.

In another embodiment the structural, neutral, zwitterionic or non-cationic lipid content of the lipid nanoparticle is composed of less than 20, 10, or 5 mol % of non-phosphatidylcholine lipids, such as DOPE (measured relative to total phosphatidylcholine, structural lipid or neutral lipid content).

In another embodiment the structural, neutral, zwitterionic or non-cationic lipid content of the lipid nanoparticle is composed of less than 20, 10, or 5 mol % of non-phosphatidylcholine lipids, such as POPC (measured relative to total phosphatidylcholine, structural lipid or neutral lipid content).

In some embodiments, the transition temperature of the structural, neutral, zwitterionic or non-cationic lipid, e.g., a phospholipid having a choline head group, is at least 20° C., 21° C., 22° C., 23° C., 24° C., 25° C., 26° C., 27° C., 28° C., 29° C., 30° C., 31° C., 32° C., 33° C., 34° C., 35° C., 36° C., 37° C. or 38° C. Without intending to be limited by any particular theory, it is believed that fusion and agglomeration of lipid nanoparticles with no hydrophilic polymer lipid conjugate (or low levels thereof) during particle formation using the mixing method described herein could be avoided by selecting a structural, neutral, zwitterionic or non-cationic lipid that is in the gel phase rather than in the disordered liquid crystalline phase at room temperature and above. The inclusion of such structural, neutral, zwitterionic or non-cationic lipid in the lipid nanoparticle may also improve blood stability after injection.

The structural, neutral, zwitterionic or non-cationic lipid content is determined based on the total amount of lipid in the lipid nanoparticle, including the sterol.

### Ionizable Lipid

The LNP of the disclosure has an ionizable lipid. The ionizable lipid may be charged at low pH and have substantially no net charge at physiological pH. This allows for electrostatic interactions between the lipid and the negatively charged nucleic acid cargo during initial formulation. Since the ionizable lipid is near neutral at physiological pH, toxicity and renal clearance are reduced. After cellular uptake by endocytosis, the acidic environment of the endosome leads to an increase in the net positive charge of the ionizable amino lipids, which promotes fusion with the anionic lipids of the endosomal membrane and subsequent membrane destabilization and release of the nucleic acid-based therapeutics into the cytoplasm to exert their effects.

In some embodiments, the LNP has an apparent pKa of between 5.0 and 7.5, between 6.5 and 7.5 or between 6.6 and 7.3. The apparent pKa is measured using a 6-(p-Toluidino)-2-naphthalenesulfonic acid (TNS) assay adapted from previous studies from other groups (Shobaki et al., 2018, International Journal of Nanomedicine, 13:8395-8410; and Jayaraman et al., 2012, Angew. Chem Int. Ed., 51:8529-8533, which are incorporated herein by reference for the purposes of determining apparent pKa). According to the method, a series of buffers are prepared spanning a pH range of 2-11 in 0.5 pH unit increments consisting of 130 mM NaCl, 10 mM ammonium acetate, 10 mM 2-(N-morpholino)ethanesulfonic acid (MES), and 10 mM HEPES. 0.15-0.2 mM of the LNP. A solution of 0.06 mM of TNS is subsequently mixed with 175 μL of the LNP at each buffered pH in triplicate in a black, polysterene 96-well plate, to yield a final concentration of 6.25 and 6 μM of lipid and TNS in each well, respectively. Fluorescence is subsequently measured using an SpectraMax™ M5 microplate reader at λₑₓ=321 nm, λₑₘ=445 nm. The fluorescence is then plotted against pH using a sigmoidal curve fit through Prism™, in which the pKa is determined to be the pH value with 50% of maximal fluorescent intensity.

In some embodiments, it is desirable to include less than 50 mol % ionizable lipid. That is, the ionizable lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the ionizable lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

As used herein, the term “ionizable cationic lipid” refers to a lipid that, at a given pH, such as physiological pH, is in an electrostatically neutral form and that accepts protons, thereby becoming electrostatically positively charged, and for which the electrostatically neutral form has a calculated logarithm of the partition coefficient between water and 1-octanol (i.e., a c Log P) greater than 8. In some embodiments, the cationic lipid has a pKa that is between 5.0 and 8.0, 5.0 and 7.5 or between 6.0 and 7.5.

In some embodiments, the ionizable cationic lipid has an amino group. In another embodiment, the ionizable cationic lipid has a single amino group that is ionizable. In some embodiments, the ionizable cationic lipid comprises a protonatable tertiary amine (e.g., pH titratable) head group. Such lipids include, but are not limited to sulfur lipids, such as MF019 described herein and DODMA. Other lipids that may be used in the practice of the disclosure include MC3- and KC2-type lipids, which are well-known to those of skill in the art. In further embodiments, the ionizable lipid is selected from one or more lipids set forth in WO 2022/246555; WO 2022/246568; WO 2022/246571; WO 2023/147657; WO2022/155728; WO 2023/215989; WO 2024/065041; WO 2024/065042; WO 2024/130421; WO 2024/065043; and U.S.2024/0294462, each incorporated herein by reference

In one embodiment, the ionizable cationic lipid has a protonatable amino head group; at least two lipophilic moieties, wherein the amino head group has a central nitrogen atom or carbon atom to which each of the two lipophilic moieties are directly bonded; each lipophilic chain has between 15 and 40 carbon atoms in total; and wherein the lipid has (i) a pKa of between 6 and 7.5; and (ii) a log P of at least 11.

Optionally, at least one of the lipophilic moieties bonded to the head group has a biodegradable group. In one non-limiting example, at least one of the lipophilic moieties has an ester group in any orientation and a sulfur atom. In one embodiment, the ionizable cationic lipid has a lipophilic moiety of the formula:

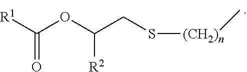

In one embodiment, R¹ and R² are, independently, linear, cyclic and/or branched optionally substituted C₃-C₂₀ alkyl and optionally with varying degrees of unsaturation; and n is 4 to 8.

In some embodiments, it is desirable to include less than 50 mol % ionizable cationic lipid in the LNP. That is, the ionizable cationic lipid content may be less than 50 mol %, less than 45 mol %, less than 40 mol %, less than 35 mol %, less than 30 mol %, less than 25 mol %, less than 20 mol %, less than 15 mol %, less than 10 mol % or less than 5 mol %.

In certain embodiments, the ionizable cationic lipid content is from 5 mol % to 50 mol % or 8 mol % to 47 mol % or 10 mol % to 50 mol % or 15 mol % to 45 mol % or 15 mol % to 35 mol % of total lipid present in the lipid nanoparticle.

The ionizable lipid component may include an ionizable anionic lipid as part of the ionizable lipid content. An example of such a lipid is cholesteryl hemisuccinate (CHEMS). Further examples of ionizable anionic lipids are described in co-pending and co-owned PCT/CA2024/050347, which is incorporated herein by reference in its entirety.

In some embodiments, the ionizable cationic lipid is not a lipidoid structure, including but not limited to C12-200 (see Khare et al., 2022, AAPS Journal, 24:8, incorporated by reference) and related structures known to those of skill in the art.

### Sterol

The LNP further includes a sterol in some embodiments. The term “sterol” refers to a naturally-occurring or synthetic compound having a gonane skeleton and that has a hydroxyl moiety attached to one of its rings, typically the A-ring.

Examples of sterols include cholesterol, or a cholesterol derivative, the latter referring to a cholesterol molecule having a gonane structure and one or more additional functional groups.

The cholesterol derivative includes β-sitosterol, 3-sitosterol, campesterol, stigmasterol, fucosterol, or stigmastanol, dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 30[N—(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5α-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol or a salt or ester thereof.

In one embodiment, the sterol is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is cholesterol and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In another embodiment, the sterol is a cholesterol derivative and is present at from 15 mol % to 50 mol %, 18 mol % to 45 mol %, 20 mol % to 45 mol %, 25 mol % to 45 mol % or 30 mol % to 45 mol % based on the total lipid present in the lipid nanoparticle.

In one embodiment, the combined (i) sterol content (e.g., cholesterol or cholesterol derivative thereof); and (ii) neutral lipid content is at least 50 mol %; at least 55 mol %, at least 60 mol %, at least 65 mol %, at least 70 mol %, at least 75 mol %, at least 80 mol % or at least 85 mol % based on the total lipid present in the lipid nanoparticle.

### Hydrophilic Polymer-Lipid Conjugate

In one embodiment, the lipid nanoparticle comprises a hydrophilic-polymer lipid conjugate capable of incorporation into the LNP. The conjugate includes a lipid or lipophilic moiety covalently attached to a polymer chain that is hydrophilic, optionally via a linker region. Examples of hydrophilic polymers include polyethyleneglycol (PEG), polyvinylpyrrolidone, polyvinylmethylether, polyhydroxypropyl methacrylate, polyhydroxypropylmethacrylamide, polyhydroxyethyl acrylate, polymethacrylamide, polydimethylacrylamide, polymethyloxazoline, polyethyloxazoline, polyhydroxyethyloxazoline, polyhydroxypropyloxazoline, polysarcosine and polyaspartamide. In one embodiment, the hydrophilic-polymer lipid conjugate is a PEG-lipid conjugate. The hydrophilic polymer lipid conjugate may also be a naturally-occurring or synthesized oligosaccharide-containing molecule, such as monosialoganglioside (G_{M1}). The ability of a given hydrophilic-polymer lipid conjugate to enhance the circulation longevity of the LNPs herein could be readily determined by those of skill in the art using known methodologies.

The hydrophilic polymer lipid conjugate may be present in the nanoparticle at 0.5 mol % to 5 mol %, or at 0.5 mol % to 3 mol %, or at 0.5 mol % to 2.5 mol % or at 0.5 mol % to 2.0 mol % or at 0.5 mol % to 1.8 mol % of total lipid. In certain embodiments, the hydrophilic polymer lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In another embodiment, the PEG-lipid conjugate is present in the nanoparticle at 0.5 mol % to 5 mol %, or at 0.5 mol % to 3 mol % or at 0.5 mol % to 2.5 mol % or at 0.5 mol % to 2.0 mol % or at 0.5 mol % to 1.8 mol % of total lipid. In certain embodiments, the PEG-lipid conjugate may be present in the nanoparticle at 0 mol % to 5 mol %, or at 0 mol % to 3 mol %, or at 0 mol % to 2.5 mol % or at 0 mol % to 2.0 mol % or at 0 mol % to 1.8 mol % of total lipid.

In one embodiment, the lipid nanoparticle has “substantially no hydrophilic polymer-lipid conjugate” or is “non-sterically stabilized”, “unshielded” or “uncoated”, meaning the lipid nanoparticle has less than 0.8 mol % total hydrophilic-polymer lipid conjugate content or other surface stabilizer content as measured based on the total lipid content of the nanoparticle as measured based on the total lipid content of the nanoparticle. In some embodiments, the hydrophilic-polymer lipid conjugate or other surface stabilizer content is less than 0.75, 0.70, 0.65, 0.60, 0.55, 0.50, 0.45, 0.40, 0.35, 0.30, 0.25, 0.20, 0.15, 0.10 mol % as measured based on the total lipid content of the nanoparticle. In further embodiments, the hydrophilic-polymer lipid conjugate or other surface stabilizer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %.

As used herein, the term “surface stabilizer” is a macromolecule, including a protein, polysaccharide or polymer, including a block copolymer, that is used to stabilize a lipid nanoparticle, and in which at least a portion (e.g., hydrophilic) is present on the surface of the lipid nanoparticle. Such molecules are employed by those of skill in the art to prevent aggregation, improve shelf life and/or improve the stability of the particle after administration, such as the circulation lifetime of the lipid nanoparticle. The term includes surface stabilizers that are known to control the size of lipid nanoparticles, such as amphiphilic polymers (e.g., block co-polymer). As would be appreciated by those of skill in the art, a hydrophobic portion of the surface stabilizer may partition in a lipophilic portion of the lipid nanoparticle.

In some embodiments, the surface stabilizer is present at less than 0.75, 0.70, 0.65, 0.60, 0.55, 0.50, 0.45, 0.40, 0.35, 0.30, 0.25, 0.20, 0.15, 0.10 mol % as measured based on the total lipid content of the lipid nanoparticle. In further embodiments, the surface stabilizer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %.

In some embodiments, the hydrophilic-polymer conjugate (e.g., a hydrophilic-polymer lipid conjugate) content is less than 0.75, 0.70, 0.65, 0.60, 0.55, 0.50, 0.45, 0.40, 0.35, 0.30, 0.25, 0.20, 0.15 or 0.10 mol % as measured based on the total lipid content of the nanoparticle. In further embodiments, the hydrophilic-polymer conjugate (e.g., a hydrophilic-polymer lipid conjugate) mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %.

In some embodiments, the amphipathic polymer content is less than 0.75, 0.70, 0.65, 0.60, 0.55, 0.50, 0.45, 0.40, 0.35, 0.30, 0.25, 0.20, 0.15, 0.10 mol % as measured based on the total lipid content of the nanoparticle. In further embodiments, the amphipathic polymer mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %. Examples of amphipathic polymers are provided in US 2021/0046192, which is incorporated herein by reference.

In some embodiments, the poloxamer content is less than 0.50, 0.45, 0.40, 0.35, 0.30, 0.25, 0.20, 0.15, 0.10 mol % as measured based on the total lipid content of the nanoparticle. In further embodiments, the poloxamer mol % content is between 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol %, 0 and 0.10 mol % or 0 and 0.05 mol %.

In further embodiments, the LNP lacks a surface stabilizer that is a protein, referred to as a protein stabilizer. This includes an apolipoprotein stabilizer, derivative or mimetic thereof (see e.g., WO 2023/233042, which is incorporated herein by reference). Such apolipoprotein may be selected from one or a combination of apo A1, apo A1-Milano, apo A2, apo A4, apo A5, apo B48, apo B100, apo C-1, apo C-1 I, apo C-111, apo C-IV, apo D, apo E, apo F, apo H, apo L and apo M. In some embodiments, the protein stabilizer content is less than 0.75, 0.70, 0.65, 0.60, 0.55, 0.50, 0.45, 0.40, 0.35, 0.30, 0.25, 0.20, 0.15 or 0.10 mol % as measured based on the total lipid content of the nanoparticle. In some embodiments, the protein stabilizer content is less than 0.45, 0.40, 0.35, 0.30, 0.25, 0.20, 0.15 or 0.10 mol % as measured based on the total lipid content of the nanoparticle. In further embodiments, the protein stabilizer content mol % content is between 0 and 0.75 mol %, 0 and 0.70 mol %, 0 and 0.65 mol %, 0 and 0.60 mol %, 0 and 0.55 mol %, 0 and 0.50 mol %, 0 and 0.45 mol %, 0 and 0.40 mol %, 0 and 0.35 mol %, 0 and 0.30 mol %, 0 and 0.25 mol %, 0 and 0.20 mol %, 0 and 0.15 mol % or 0 and 0.10 mol %. Since lipid nanoparticles can adsorb proteins after administration, the protein content is measured in vitro prior to administration.

Alternatively, in some embodiments a lipid nanoparticle preparation lacks or has low levels thereof of one or more stabilizing agents, which includes a surface stabilizer as described above and a cryoprotectant. In some embodiments, the lipid nanoparticle preparation has less than 2 w/v, 1.75 w/v, 1.50 w/v, 1.25 w/v, 1.00 w/v, 0.75 w/v, 0.50 w/v, 0.25, 0.10 or 0.05 w/v of one or more cryoprotectants in the preparation. In some embodiments, a lipid nanoparticle preparation having a plurality of LNPs has low levels or lacks glycerol and/or propylene glycol as a cryoprotectant, such as at concentration levels less than 2 w/v, 1.75 w/v, 1.50 w/v, 1.25 w/v, 1.00 w/v, 0.75 w/v, 0.50 w/v, 0.25, 0.10 or 0.05 w/v in the preparation.

In another embodiment, the lipid nanoparticle is “PEG-less”, meaning that the lipid nanoparticle has no detectable amounts of polyethylene-glycol lipid conjugate.

Examples of lipid nanoparticles with low levels or no hydrophilic polymer lipid conjugate or other surface stabilizer that can be used in the practice of the disclosure are described in co-owned and co-pending U.S. provisional patent applications 63/556,432 and 63/588,167, which are incorporated herein by reference. Such lipid nanoparticles are also described in Examples 1-3 herein.

### Additional Lipid Components

The LNP may comprise additional lipid components besides those described above (neutral lipid, cholesterol, ionizable cationic lipid and the optional hydrophilic polymer-lipid conjugate). Without limitation, such additional lipid components may be present at less than 10 mol %, 9 mol %, 8 mol %, 7 mol %, 6 mol %, 5 mol %, 4 mol %, 3 mol %, 2 mol %, 1 mol % or 0.5 mol % (relative to total lipid in the LNP). Such additional lipids include lipids comprising a targeting moiety, charged lipid (cationic or anionic lipid that is charged at physiological pH) or other lipid components such as vitamins (e.g., tocopherol). In some embodiments, the LNP consists essentially of neutral lipid, cholesterol, ionizable cationic lipid and the optional hydrophilic polymer-lipid conjugate, meaning any additional lipid is present at less than 5 mol % measured relative to total lipid in the LNP.

The LNP may comprise a targeting moiety for targeting the lipid nanoparticle to T cells. An antibody conjugated LNP may be targeted to receptors present on T cells, such as CD4 and CD8. The targeting moiety may be conjugated directly to a lipophilic moiety that resides in the LNP membrane or may be conjugated to the distal end of a hydrophilic polymer, if present. Examples of LNPs with targeting moieties are described in co-owned and co-pending PCT/CA2023/051632, which is incorporated herein by reference.

In one embodiment, the LNP lacks a ligand-lipid conjugate for targeting to T cells. In such embodiments, the ligand-lipid conjugate is undesirable as it may induce an immune response. Instead, targeting may be achieved by the inherent extrahepatic delivery properties of the lcLNP™ due to elevated phosphatidylcholine content. Thus, in some embodiments the ligand-lipid conjugate is present at less than 2 mol %, less than 1.5 mol %, less than 1 mol %, less than 0.5 mol %, less than 0.25 mol % or is 0 mol %.

In another embodiment, the additional component may include an anionic phospholipid, such as phosphatidylserine, and/or an ionizable anionic lipid. An example of such a lipid is cholesteryl hemisuccinate (CHEMS). Further examples of ionizable anionic lipids are described in co-pending and co-owned PCT/2024/050347, which is incorporated herein by reference in its entirety.

Alternatively or additionally, the additional lipid component may include permanently charge cationic lipid, including lipids with a quarternary ammonium cation (e.g., DOTMA, DOSPA, DDAB and DOTAP) or a zwitterionic, anionic lipid, such as phosphatidylserine. Such permanently charged lipids, in some examples, are most advantageously present at less than 10 mol %, 9 mol %, 8 mol %, 7 mol %, 6 mol %, 5 mol %, 4 mol %, 3 mol %, 2 mol %, 1 mol %, 0.5 mol % or 0.25 mol % relative to total lipid content.

### Nanoparticle Preparation and Morphology

Delivery vehicles incorporating the cargo can be prepared using a variety of suitable methods, such as a rapid mixing/ethanol dilution process. Examples of preparation methods are described in Jeffs, L. B., et al., 2005, Pharm Res, 22(3):362-72; and Leung, A. K., et al., 2012, The Journal of Physical Chemistry. C, Nanomaterials and Interfaces, 116(34): 18440-18450, each of which is incorporated herein by reference in its entirety.

Without being bound by theory, the mechanism whereby a lipid nanoparticle comprising encapsulated cargo can be formed using the rapid mixing/ethanol dilution process can be hypothesized as beginning with formation of a dense region of hydrophobic mRNA-ionizable lipid core at low pH (e.g., pH 4) surrounded by a monolayer of helper lipid/cholesterol that fuses with smaller empty vesicles as the pH is raised due to the conversion of the ionizable cationic lipid to the neutral form. As the proportion of bilayer helper lipid increases, the bilayer lipid progressively forms blebs and the ionizable lipid migrates to the interior hydrophobic core. At high enough helper lipid contents, the exterior bilayer preferring helper lipid can form a complete lipid layer, such as a continuous or discontinuous bilayer, around the interior trapped volume.

The LNP may comprise a “core” region. It has been observed that the LNP core is non-homogeneous in that it includes both an electron dense region and an aqueous portion or compartment as visualized by cryo-EM microscopy. In some embodiments, the core may be characterized as non-solid. Without being limiting, the electron dense region within the core may be partially surrounded by the aqueous portion or compartment within the enclosed space as observed by cryo-TEM. The aqueous portion may form a distinct aqueous region or compartment within the lipid nanoparticle. In other words, it is believed that the aqueous portion or compartment is not merely a hydration layer.

In one embodiment, at least one about fifth of the core (trapped volume) contains the aqueous portion or compartment, and in which the electron dense region within the core is partially contiguous with the lipid layer comprising the bilayer, as determined qualitatively by cryo-EM. In another embodiment, at least one about quarter of the core contains the aqueous portion or compartment, and in which the electron dense core is either partially contiguous with the lipid layer comprising the bilayer, as determined qualitatively by cryo-EM. In a further embodiment, at least one about one third of the core contains the aqueous portion or compartment, and in which the electron dense region is either partially contiguous with the lipid layer comprising the bilayer, as determined qualitatively by cryo-EM. In another embodiment, at least one about one half of the core contains the aqueous portion or compartment, and in which the electron dense core is partially contiguous with the lipid layer comprising the bilayer, as determined qualitatively by cryo-EM.

In another embodiment, the electron dense region of the LNP surprisingly appears to be completely surrounded by the aqueous portion of the core as visualized by cryo-TEM microscopy. This morphology is observed in a single plane and a portion of the electron dense region as observed is contiguous with the lipid layer (e.g., bilayer) but cannot be seen since this portion is not within the plane that can be visualized.

In one embodiment, the electron dense region is generally spherical in shape. In another embodiment, the electron dense region is hydrophobic.

The lipid nanoparticles herein may exhibit particularly high trapping efficiencies of mRNA. Thus, in one embodiment, the trapping efficiency is at least 50, 55, 60, 65, 70, 75, 80, 85 or 90%.

In another embodiment, the cargo and cationic ionizable lipid are present in the electron dense region. In a further embodiment, the helper lipid is present in the lipid layer comprising the bilayer.

The lipid nanoparticle may comprise a single bilayer or may be a combination of a bilayer and a monolayer in some embodiments. In one embodiment, the lipid layer is a continuous bilayer that surrounds the core.

In certain embodiments the electron dense region of the core is separated from the lipid layer comprising the bilayer by the aqueous portion or compartment. For example, the disclosure provides a lipid nanoparticle preparation comprising a plurality of lipid nanoparticles in which at least 20%, 30%, 40%, 50%, 60% or 70% of the particles as determined by cryo-EM microscopy have a core with an electron dense region and an aqueous portion or compartment and in which the aqueous portion or compartment is partially surrounded by the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have an elongate shape (e.g., generally oval-shaped) as determined qualitatively by cryo-EM microscopy. In this latter embodiment, the electron dense region of the core may be partially surrounded the aqueous space as visualized by cryo-EM microscopy.

In one embodiment, the lipid nanoparticle is part of a preparation of lipid nanoparticles, and wherein the electron dense region of at least 20% of the lipid nanoparticles are either (i) enveloped by the aqueous portion, or (ii) is partially surrounded by the aqueous portion and wherein a portion of a periphery of the electron dense region is contiguous with the lipid layer, as visualized by cryo-EM microscopy in a single plane.

In certain embodiments, the disclosure provides a lipid nanoparticle preparation comprising a plurality of lipid nanoparticles in which generally at least 10%, 20%, 30%, 40%, 50%, 60% or 70% of the particles as determined by cryo-EM microscopy have a core with an electron dense region that is contiguous with the lipid layer comprising the bilayer as visualized by cryo-EM microscopy.

In another embodiment, and without being limiting, the disclosure provides a lipid nanoparticle preparation comprising a plurality of lipid nanoparticles in which generally at least 10%, 20%, 30%, 40%, 50%, 60% or 70% of the particles have a core comprising an electron dense region that appears to be surrounded or enveloped by a continuous aqueous space disposed between the lipid layer (e.g., bilayer) and the electron dense region, as visualized in one plane by cryo-EM microscopy.

LNPs are visualized by cryo-TEM as described in co-owned and co-pending WO 2022/251959, incorporated herein by reference.

In another embodiment, the polydispersity index (PDI) of the LNP preparation is less than 0.50, 0.45, 0.40, 0.35, 0.30, 0.25, 0.2, 0.15, 0.12 or 0.10.

In another embodiment, the particle size distribution is such that at least 90% of the particles in the LNP preparation of the disclosure have a diameter of between 40 and 150 nm or between 40 and 140 nm or between 45 and 150 nm or between 50 and 150 nm or between 50 and 120 nm or between 50 and 140 nm.

The lipid nanoparticles herein may exhibit particularly high encapsulation efficiencies of nucleic acid. As used herein, the term “encapsulation,” with reference to incorporating the cargo (e.g., nucleic acid) within a lipid nanoparticle refers to any association of the cargo with any lipid component or compartment of the lipid nanoparticle, including a lipophilic or the aqueous portion. In one embodiment, the cargo is present at least in the core of the LNP.

In one embodiment, the encapsulation efficiency is at least 50, 55, 60, 65, 70, 75, 80, 85, 90% or 92%. The encapsulation efficiency of the cargo is determined as set forth in the Materials and Methods section in the Examples herein.

Embodiments of the present disclosure also provide lipid nanoparticles described according to the molar ratio between the positively charged amine groups of the amine lipid (N) and the negatively charged phosphate groups (P) of the oligonucleotide to be encapsulated. This may be mathematically represented by the equation N/P. In one embodiment, the N/P ratio of the lipid nanoparticle is between 2 and 15, between 3 and 15, between 4 and 15 or between 4.5 and 10 or between 5 and 10 or between 5.5 and 8.

In one embodiment, the N/P ratio of the lipid nanoparticle is at least 2, 3, 4, 4.25, 4.50, 4.75, 5.0, 5.25, 5.5, 5.75, 6.0 or 6.25. The upper limit may be 15, 14, 13, 12, 11, 10, 9 or 8. The disclosure also encompasses a combination of any two of the upper and lower limits.

In one embodiment, the lipid nanoparticle has a weight nucleic acid/micromole of total lipid that is 0.05:1 to 1:1. In one embodiment, the lower limit is 0.06:1, 0.08:1, 0.10:1, 0.12:1, 0.14:1, 0.16:1, 0.18:1, 0.20:1, 0.22:1, 0.24:1, 0.26:1, 0.28:1, 0.30:1, 0.32:1, 0.34:1, 0.36:1, 0.38:1 or 0.40:1 weight nucleic acid/micromole of total lipid. In another embodiment, the upper limit is 0.80:1, 0.82:1, 0.84:1, 0.86:1, 0.88:1, 0.90:1, 0.92:1, 0.94:1, 0.96:1 or 0.98:1 weight nucleic acid/micromole of total lipid. The disclosure also encompasses a combination of any two of the upper and lower limits.

In one embodiment, the mRNA copy number/LNP is 1-10 or 4-8.

Improved Delivery of Nucleic Acid to T Cells Using lcLNP™

In one embodiment, the lipid nanoparticle exhibits at least a 5%, 10%, 30% or 40% increase in gene editing of a T cell locus, for example T cell receptor α constant (TRAC), as measured in a T cell ex vivo relative to an otherwise identical baseline LNP control as measured by INDEL % and knockout scores and using the materials and methods of Example 2. In another embodiment, the lipid nanoparticle exhibits at least a 5%, 10%, 30% or 40% decrease in T cell receptor in a T cell in vitro relative to an otherwise identical baseline LNP control as measured by flow cytometry using the protocol of Example 2.

It has also been shown herein that the lipid nanoparticle compositions exhibit improved delivery of nucleic acid expressing a peptide, polypeptide or protein to a T cell in vivo. In an embodiment, the T cell is a CD4 T cell, CD8 T cell, or a combination thereof.

As used herein, “expression” of nucleic acid (e.g., mRNA or pDNA) refers to translation of the nucleic acid into a peptide (e.g., an antigen), polypeptide, or protein and also can include, as indicated by context, the post-translational modification of the peptide, polypeptide or fully assembled protein (e.g., enzyme). The nucleic acid (e.g., mRNA or pDNA) may encode for a protein or peptide in a gene editing complex. The polypeptide or protein encoded by the nucleic acid may comprise one or more functional domains, optionally linked by spacer regions.

In one embodiment, the lipid nanoparticle exhibits at least a 5%, 10%, 30% or 40% increase in gene expression of a cargo nucleic acid in vivo as measured in an T cell relative to baseline LNP control.

As described in Example 4, the LNP of the disclosure may provide improved in vivo nucleic acid delivery to T cell than previous baseline formulations for nucleic acid delivery. Whether or not a lipid particle exhibits such enhanced delivery to a T cell sub-population can be determined by biodistribution studies in an in vivo mouse model. In such embodiments, enhanced green fluorescent protein (eGFP) may be used to detect nucleic acid expression in a given T cell population. In particular, according to such embodiments, LNP mRNA systems are prepared encapsulating mRNA coding for eGFP and biodistribution and GFP expression in cell populations in T cells are evaluated using flow cytometry following systemic administration. It will be understood that measurement of GFP expression is used as a proxy to assess whether a given LNP falls within the scope of the invention. Any clinical formulations meeting these expression criteria will typically not contain a reporter such as GFP, although animal studies based on GFP expression as described herein can be used to determine whether or not a lipid nanoparticle possesses the improved T cell targeting. In other words, the test using an animal model to assess improved delivery to a T cell relative to a baseline LNP, will typically involve obtaining a clinical LNP formulation with a cargo having a therapeutic or prophylactic effect on a subject (typically human) and conducting tests on an LNP having the same lipid components to determine if the LNP formulation itself exhibits improved T cell targeting. For example, to determine if a given clinical LNP formulation exhibits the improved T cell delivery, the cargo could be replaced with mRNA coding for eGFP and expression in vivo compared to the baseline formulation.

To assess whether a given lipid nanoparticle exhibits an increase in gene expression in a relevant T cell at 4 hours, 12 hours, 24 hours, 48 hours or 3 days post-injection, the mRNA-LNP of the disclosure is compared to the baseline formulation of Example 4. The two LNPs being compared are subjected to the same experimental methods and materials to determine in vivo expression as set forth in Example 4.

In one embodiment, the lipid nanoparticle exhibits at least a 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290% or 300% increase in gene expression of an encapsulated mRNA encoding enhanced GFP (eGFP) as measured in vivo in T cell population sub-set at 4, 24 hours and/or 3 days post-injection as compared to a lipid nanoparticle encapsulating eGFP-mRNA with a baseline formulation of nor-MC3/DSPC/cholesterol/PEG-lipid at 50/10/38.5/1.5; mol:mol, wherein the protein expression from the nucleic acid is measured in a mouse model by detection of the eGFP translated from the mRNA. The measurement is carried out using flow cytometry and protein expression from the nucleic acid as determined by quantifying % positive cell counts in a given T cell subset as set forth in Example 4. The percentage increase is determined by comparing the percentage positive cells (i.e., detection of eGFP) in a given T cell type in the bone marrow or blood and comparing this percentage to the percentage of positive cells resulting from injecting the baseline formulation using otherwise identical materials and methods.

### Modified T Cells to Provide a Therapeutic, Prophylactic or Ameliorative Effect In Vivo

In one embodiment, the T cell is modified by the LNP herein to produce a CAR T cell. In such embodiments, the LNP comprises a nucleic acid that encodes a chimeric antigen receptor (CAR). As used herein, the term “nucleic acid encoding CAR,” includes any nucleic acid (e.g., RNA, DNA or hybrids thereof), that expresses the CAR (chimeric antigen receptor) and that is capable of encapsulation in the LNP herein.

The nucleic acid that encodes for the CAR to the T cell results in expression of the CAR. The transfected CAR-T cells express chimeric antigen receptor (CAR) on their surface that targets them in vivo to receptors on target cells of interest. Upon binding to the surface antigen, the CAR T cells become activated and exert a desired therapeutic and/or prophylactic immune response against the target cell.

In one embodiment, the CAR is encoded by mRNA encapsulated by the lipid nanoparticle. The mRNA is delivered to a T cell ex vivo or in vivo and subsequently expresses CAR and the CAR is inserted into the membrane of the T cell. In another embodiment, the CAR is encoded by nucleic acid (e.g., a transgene or a cassette) that is inserted into the T cell receptor (TCR) alpha constant (TRAC) locus of the T cell. In some embodiments, the nucleic acid is inserted into the genome of the T cell using any suitable editing cargo, including those described above.

In some embodiments, the integration of nucleic acid expressing CAR into the TRAC locus of a T cell may be facilitated by nuclease assisted homology directed repair (HDR). HDR-enhancing substances are optionally used to increase integration rates using gene editors. Non-limiting examples of HDR enhancers include ALT-R HDR enhancer Version 1 and Alt-R HDR enhancer Version 2 (available from Integrated DNA Technologies™; see also Kath et al., 2021, Molecular Therapy, Methods & Clinical Development, 25:311-330). Cyclic GMP-AMP synthase (cGAS) inhibitors may also be used to improve expression of integrated nucleic acid expressing CAR. The cGAS-STING signaling pathway, when activated, leads to production of type I interferons (IFNs) and the expression of interferon stimulated genes (ISGs), which can suppress expression levels of transfected genes (Fu et al., 2020, iScience, 23:101026). Inhibitors may be used to reduce or suppress signaling cascades that suppress transgene expression. Non-limiting examples are cGAS-specific small molecular inhibitors that reduce double stranded DNA triggered interferon expression, including: scaffold G compounds, such as G140 and G150 (Lama et al., 2019, Nature Communications 10:2261); cyclopeptides that bind to the DNA binding site of cGAs, such as XQ2B (Wang et al., 2023, Nature Communications, 14:6132); and flavonoids, such as baicalein and baicalin (Li et al., 2023, Bioorganic Chemistry, 140:106802). Further examples of cGAS inhibitors include those described in WO 2017/176812, which is incorporated herein by reference. In further non-limiting examples, a combination of cGas inhibitors and HDR enhancers may be used to improve transgene expression.

In another embodiment, a nucleic acid is integrated as a TRAC knock-in using SeLection by Essential gene Exon Knock-in; SLEEK) as described in Allen et al., 2024, Nature Biotechnology 42:458-469, which is incorporated herein by reference.

Whether expressed by mRNA cargo or a nucleic acid introduced at a loci by “knock-in”, the CAR is subsequently inserted into the T cell membrane. The CAR includes an extracellular antigen recognition moiety and a transmembrane domain for insertion in the T cell membrane. An internal domain or domains may be linked to the transmembrane domain or form part of the intracellular region of transmembrane domain. As discussed below, such internal (intracellular) domain may include one or more signaling domains, which in some embodiments improve the ability of the T cells to proliferate in vivo.

The antigen recognition moiety includes a variety of known structures for binding to an antigen of interest. For example, the antigen recognition moiety may include an antigen-recognizing single chain variable fragment (scFv) derived from an antibody sequence. The scFv may comprise a variable light (VL) and variable heavy (VH) regions of the scFv against the cell surface antigen of interest. A variety of linker regions may optionally link the VL and VH domains. In some non-limiting examples, the linker region comprises repeating glycine and serine residues. Alternatively, in some embodiments, the CAR antigen recognition moiety may include a single domain or multiple domains. Non-limiting examples of single domains include VHH of camelid antibodies, natural ligands or artificial protein binding constructs.

In some embodiments, a hinge region links the antigen recognition moiety to the transmembrane domain. In some embodiments, the hinge region (also referred to as a “spacer”) is sufficiently flexible to facilitate binding of the antigen recognition moiety to the surface antigen on the target cell. As would be appreciated by those of skill in the art, a variety of hinge regions may be employed in the practice of the disclosure. In some examples, the hinge is derived from CD28 and/or CD8.

The transmembrane domain inserts into the bilayer of the T cell and may function to facilitate molecular interactions between CARs. In some embodiments, the transmembrane domain may facilitate the formation of dimers or trimers between CARs. A co-stimulatory domain may include, without limitation, a CD28, 4-1BB, OX40, CD27 and/or inducible T cell co-stimulator (ICOS). In some embodiments, two costimulatory domains are included in the CAR.

In one embodiment, the CAR comprises a distal, intracellular CD3ζ cytoplasmic domain. In some examples, the intracellular CD3ζ cytoplasmic domain has three immunoreceptor tyrosine-based activation motifs (ITAMs) which signal upon phosphorylation. Such CARs may possess enhanced T cell function, and in some embodiments may include an additional protein molecule, causing production of cytokines or possess additional receptors such as costimulatory ligands. Examples of CARs known to those of skill in the art include TRUCKs (T cells Redirected for Universal Cytokine Killing) or armored CARs. (See Larson and Maus, 2021, Nat Rev Cancer, 21(3):145-161, which is incorporated herein by reference).

The CAR T cells are activated upon binding of the antigen recognition domain to the antigen on the target cell. Such activation may cause clustering and/or immobilization of the CAR.

In those embodiments in which CD3ζ chains are present, phosphorylation of ITAM domains on the CD3ζ chain may initiate signaling through the tyrosine kinase ζ-associated protein of 70 kDa (ZAP70). This initiates a T cell effector response including proliferation, release of cytokines, metabolic alterations, and cytotoxicity.

In some embodiments, the activated CAR T cells may exert a cytotoxic effect through secretion of granzyme and/or perforin. Alternatively, or additionally, death receptors are utilized, based on activation of downstream molecules such as BH3-interacting domain death agonist (BID) and FAS-associated death domain protein (FADD). As would be appreciated by those of skill in the art, cellular signaling from the internal domain or domains is dependent on the specific function of the domain chosen and can be modulated by introducing mutations thereof. The antigen recognition domain may bind to an antigen on any target cell of interest. This may include an antigen on the surface of a tumour. Examples of target antigens on tumours include CD19, CD22 and B-cell maturation antigen (BMCA).

In some embodiments, the cancer for treatment by CAR T includes hematological malignancies, such as relapsed acute B cell leukemia, aggressive B cell lymphoma, 1, 2 and treatment-refractory multiple myeloma. In some embodiments, CAR T cells can be used to treat solid tumors.

While CAR T cell therapy in some embodiments is used to treat a cancer, other diseases and conditions may be treated by CAR T. This includes, without limitation, cardiac fibrosis (see US 2023/02035338, incorporated herein by reference) or autoimmune diseases.

In some embodiments, the T cell is modified to improve CAR T cell therapy. For example, gene editing of a T cell may be used to ameliorate CAR T cell dysfunction (e.g., T cell exhaustion), modulate cytokine production or knock in CAR cassettes at specific genomic locations. In another embodiment, editing cargo can be used to produce allogeneic CAR T cells. (See Dimitri et al., 2022, Molecular Cancer, 21, article No. 78, which is incorporated herein by reference). In another embodiment, editing systems or siRNA could be used to reduce or abrogate expression of endogenous T cell receptors to reduce the risk of graft-verses-host-disease (GVHD). In some embodiments, expression of ap TCRs on the T cell surface is reduced or eliminated through genetic knockout of exons of the TCRα constant (TRAC) and/or TCRβ constant 1 (TRBC1) or 2 (TRBC2) loci (see Example 2 herein).

T cell function can be modulated to down regulate specific genes using siRNA for therapy to treat cancer, inflammation, autoimmunity and viral infections. The use of siRNA to downregulate genes in T lymphocytes may be used to treat leukocyte associated disorders (Ramishetti et al., 2015, ACS Nano 9, 7, 6706-6716). In one non-limiting example, siRNA could be used to downregulate GATA3 in activated TH2 cells to treat inflammatory disease, such as asthma (Keil et al., 2020, Nanomed Nanobiotechnol, 12:e1634).

### Administration of LNPs and Pharmaceutical Formulations Thereof

In some embodiments, the lipid nanoparticle comprising nucleic acid is part of a pharmaceutical composition and is administered to treat and/or prevent a disease condition. The treatment may provide a prophylactic (preventive), ameliorative or a therapeutic benefit. The pharmaceutical composition will be administered at any suitable dosage.

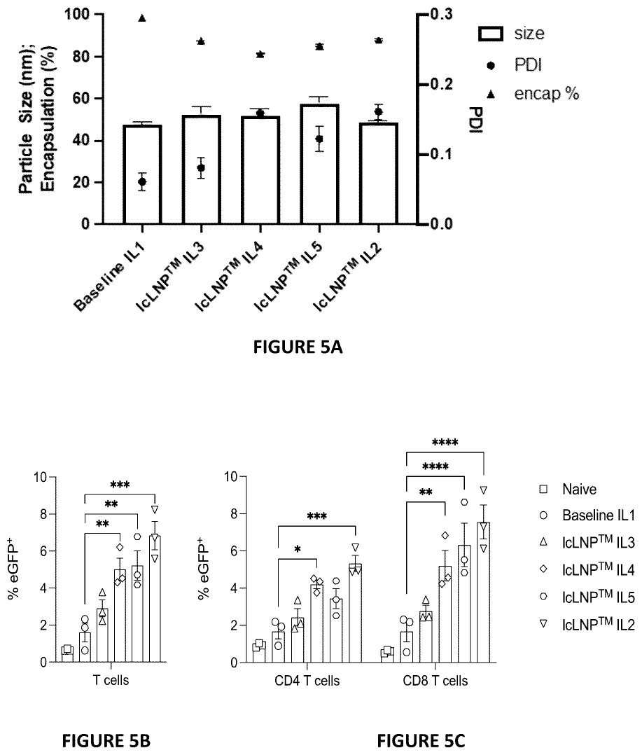

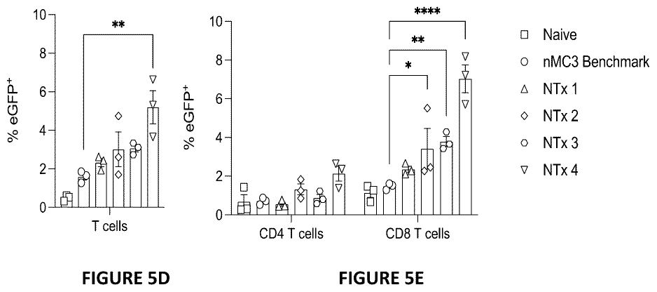

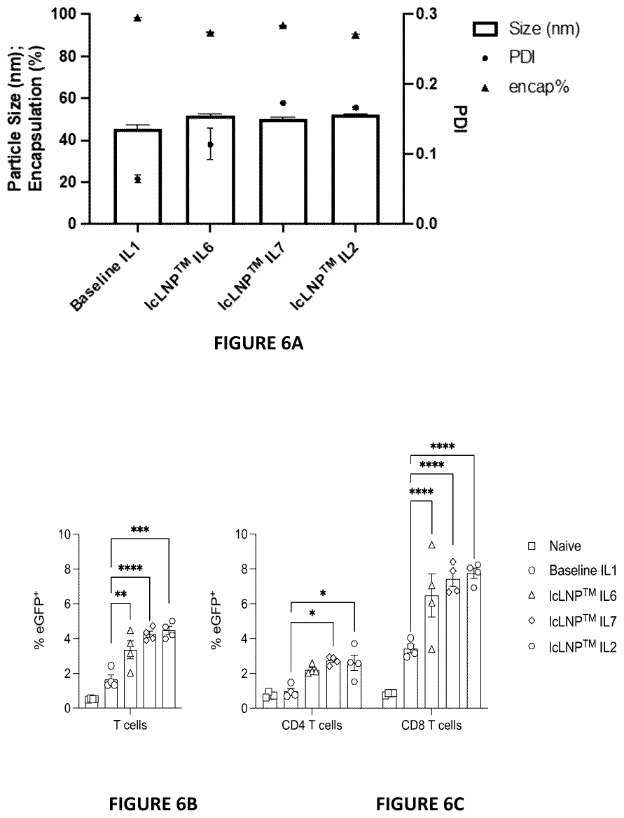

In one embodiment, the LNPs herein are injected intravenously to a subject and are targeted to extrahepatic organs or tissues, including the vascular system (e.g., blood). As discussed, the inventors have found that LNPs with elevated levels of a neutral or zwitterionic lipid (e.g., a phosphatidylcholine lipid or other structural lipid) have improved extrahepatic biodistribution. In particular, flow cytometry has revealed that T cells have increased expression of mRNA relative to a baseline formulation at 24 hours post-administration (FIGS. 5 and 6). Advantageously, in some embodiments, such extrahepatic targeting can be achieved without a targeting ligand, thereby avoiding an unwanted immune response against the ligand.

In another embodiment, the modified T cells are produced ex vivo. In such embodiments, T cells are obtained from a subject (e.g., patient) or a donor thereof, contacted with the LNP herein to produce the modified T cells and then administered to the subject. The T cells can be derived autologously from T cells in a subject's own blood or allogeneically from a donor. The T cells obtained from the subject or donor may be selected from CD4+, CD8+ or a combination thereof. In some embodiments, leukocytes are obtained by leukocyte apheresis and peripheral blood mononuclear cells are separated and collected. In some embodiments, certain populations of T cells are stimulated to proliferate and after proliferation and purification, the cells are contacted with the LNP ex vivo.

Thus, in some embodiments of the disclosure, the LNP is administered to a subject and T cells are modified in vivo after administration. Such embodiment is advantageous as it avoids isolating T cells from the subject or a donor. Improved targeting of the LNP to extrahepatic tissues or organs advantageously provides improvements in delivery to T cell populations that reside in, for example, blood, spleen, bone marrow and/or the lymphatic system. In one embodiment, the LNP is part of a pharmaceutical composition administered parenterally, i.e., intra-arterially, intravenously, subcutaneously or intramuscularly. In yet a further embodiment, the pharmaceutical compositions are for intraosseous injection (IO).

The pharmaceutical composition provides a preventative, therapeutic or ameliorative effect and comprises pharmaceutically acceptable salts and/or excipients.

The composition described herein may be administered to a subject such as a patient. The term subject as used herein includes a human or a non-human subject.

The examples below are intended to illustrate the preparation of specific lipid nanoparticle nucleic acid preparations and properties thereof but are in no way intended to limit the scope of the invention.

### EXAMPLES

### Materials and Methods

### LNP Preparation

The LNPs were prepared by dissolving mRNA in 25 mM sodium acetate, pH 4.0, while the lipid components at the mole % specified were dissolved in absolute ethanol. The lipids in ethanol and the eGFP mRNA in buffer were combined in a 1:3 volume by volume ratio using a t-junction with dual-syringe pumps. The solutions were pushed through the t-junction at a combined flow rate of 20 mL/min (5 mL/minute for the lipid-containing syringe, 15 mL/minute for the mRNA-containing syringe). The lcLNP™-PEGless formulations were made using a 1/5 dilution with 25 mM sodium acetate after T-mixing. The mixture was subsequently dialyzed overnight against at least ~100 volumes of 1× phosphate buffered saline, pH 7.4 using Spectro/Por dialysis membranes (molecular weight cut-off 12 000-14 000 Da). The LNPs were concentrated as required with an Amicon Ultra™ 10 000 MWCO (molecular weight cut-off), regenerated cellulose concentrator.

Encapsulation efficiency was calculated by determining unencapsulated mRNA content by measuring the fluorescence upon the addition of RiboGreen™ to the mRNA-LNP (Fᵢ) and comparing this value to the total mRNA content that is obtained upon lysis of the LNP by 2% Triton X-100 (Fₜ): % encapsulation=(Fₜ−Fᵢ)/Fₜ×100.

The particle size and polydispersity index (PDI) were characterized using a Zetasizer Nano ZS™.

### T7 Endonuclease Detection Assay

To assess the efficiency of TCR genomic cleavage, the GeneArt Genomic Cleavage Detection Kit (Invitrogen) was utilised. 48 hours post LNP treatment, genomic DNA extraction was performed using DNeasy Blood and Tissue Kit (QIAGEN). PCR was performed to amplify a 600 bp region covering the TCR cut site and purified using Monarch PCR & DNA Cleanup Kit (NEW ENGLAND Biolabs). 60 ng of DNA was heated to 95° C. for 5 minutes before cooling to 25° C. to generate heteroduplex DNA. The DNA was then incubated for 1 hour at 37° C. with Detection enzyme before being subject to agarose gel electrophoresis.

### Interference of CRISPR Editing (ICE)

For assessment of INDEL and knockout efficiency, the PCR amplified and purified DNA samples used for the T7 endonuclease detection assay were sent to Source Bioscience for Sanger sequencing. The resulting Sanger sequencing data was analyzed using Synthego's ICE algorithm, providing the frequency of INDELs (INDEL %) and the contribution which will produce a frameshift and therefore a functional knockout (Knockout score).

### Flow Cytometry In Vivo Studies

The LNPs at the eGFP mRNA concentration of 1.0 mg/mL were injected intravenously (i.v.) in mice at a volume using the formula weight of the mouse (in grams)*10 μL. Bone marrow and blood was harvested 24 hours after the LNP injections.

The bone marrow was harvested and processed into a single cell suspension. In particular, the mice were anesthetized with 5% isoflurane until reflex was lost and then exposed to CO₂ with 1% air. The marrow was isolated from the femur by centrifugation of the bone for 30 s at 3,810 g and resuspended in FACS buffer (1× sterile PBS (pH 7.4), 2.5 mM ethylenediaminetetraacetic acid (EDTA), 0.05% (w/v) sodium azide (NaN₃), 2% (v/v) heat-inactivated fetal bovine serum (HI-FBS)). The bone marrow was then washed once in FACS buffer before final resuspension.

After isolation, the bone marrow and/or blood cells were stained. One to three million cells were added to a well of 96-well round bottom plates and the volume in each well was increased to 150 μL using FACS buffer. Cells were centrifuged at 484 g at 4° C. for 5 minutes and the liquid was discarded. Subsequently, cells were incubated with Fc block and then a solution containing staining antibodies for 45 minutes. Cells were centrifuged at 484 g at 4° C. for 5 minutes and the liquid was discarded. The volume was increased to 150 μL and cells were centrifuged at 484 g at 4° C. for 5 minutes and the liquid was discarded twice. A volume of 150 μL of propidium iodide (PI) was added at a 1:5,000 dilution (1 mg/mL stock) and the stained, single cells were introduced to a flow cytometer (Cytoflex™, Beckman Coulter™). Single colour set-ups were used to generate the compensation matrix which was applied to all the samples.

The blood was harvested and processed into a single cell suspension. In particular, the mice were anesthetized with 5% isoflurane until reflex was lost and then exposed to CO₂ with 1% air. The blood was harvested via immediate cardiac puncture and added to 0.5 mL of 0.5M EDTA solution. The blood-EDTA solution was then transferred to 12 mL of pre-warmed 1×RBC Lysis buffer and incubated in a 37 C water bath for 5 minutes. Cells were then centrifuged at 484 g at 4° C. for 5 minutes and the liquid was discarded. Blood cells were then washed in FACS buffer (1× sterile PBS (pH 7.4), 2.5 mM ethylenediaminetetraacetic acid (EDTA), 0.05% (w/v) sodium azide (NaN₃), 2% (v/v) heat-inactivated fetal bovine serum (HI-FBS)) and resuspended in a final volume of FACS buffer.

The flow cytometry data was analyzed using FlowJo™ version 10 (Becton Dickinson™ & Company (BD)). Corresponding T cell sub-sets were identified based on an appropriate gating scheme.

Ex Vivo eGFP, TCR and CD3 Flow Cytometry

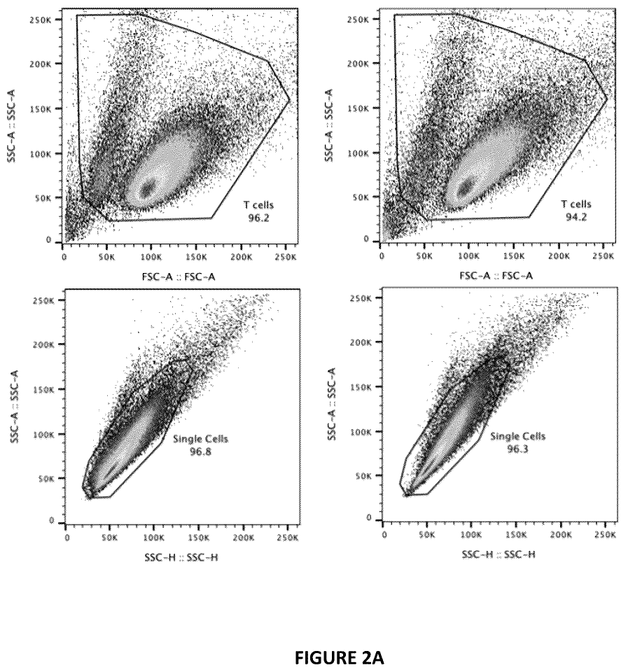

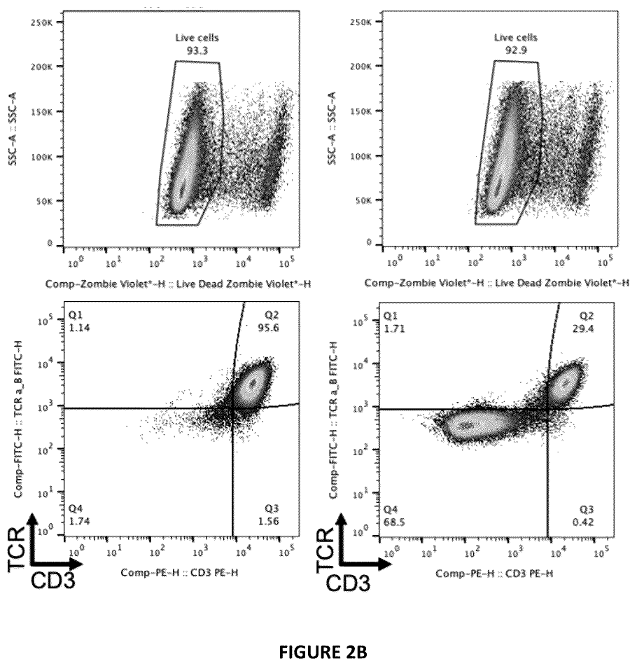

For ex vivo analysis of GFP or TCR and CD3 expression, purified CD4+ or CD8+ T cells (Stem Cell Technologies) were stained for flow cytometry analysis. 1 million cells were centrifuged for 5 minutes at 300×g and washed with FACS buffer (5% FBS, 2 mM EDTA in 1× sterile PBS (pH 7.4)) before staining in 100 μL FACS buffer for 30 minutes at 4° C. with the following reagents: Zombie Violet viability dye, PE anti-human CD3, Alexa Fluor 488 anti-human TCRα/β. Cells were washed 2× in FACS buffer before acquisition on a BD FACSMelody™ flow cytometer. An ex vivo representative flow cytometry gating strategy is provided in FIGS. 2A and 2B.

The bar graphs for the percentage of eGFP positive (eGFP⁺) cells were generated using Prism™ version 8 (GraphPad™) software.

### Ex Vivo GFP Knock-Out Knock-In

For GFP knock-in studies, LNPs were prepared at a 1:1:3 weight ratio of Cas9 mRNA:TCR gRNA:GFP HDR DNA. 1×10⁶ Primary CD4+ cells were treated with an RNA dose of 2.5 μg for 48 hours with or without co-treatment with a HDR enhancer (M3814). Cells were expanded and analysed for TCR KO and GFP knock-in (KI) at 7 days post LNP treatment. For flow cytometry analysis, 1×10⁶ cells were centrifuged for 5 minutes at 300×g and washed with FACS buffer (5% FBS, 2 mM EDTA in 1× sterile PBS (pH 7.4)) before staining in 100 μl FACS buffer for 30 minutes at 4° C. with the following reagents: Zombie Violet viability dye, APC anti-human TCRa/b. Cells were washed 2× in FACS buffer before acquisition on a BD FACSMelody™ flow cytometer. The percentage of TCR KO and GFP KI were calculated from the preceding viable cell population.

### Example 1: MRNA Gene Expression in T Cells Increases Significantly Using High Phosphatidylcholine LNPs Relative to a Baseline Formulation

The effect of increasing the amount of DSPC from 10 mol % to 50 mol % in an LNP containing enhanced green fluorescent protein mRNA (“eGFP mRNA”) cargo was evaluated in primary human CD8+ T-cells ex vivo.

In particular, the following eGFP mRNA formulations (reported in mol %) comprising ionizable lipid (as indicated), DSPC and cholesterol (no PEG) were compared in this example.

TABLE 1
Formulations examined ex vivo containing eGFP mRNA

| | Percent | | Ionizable lipid citation |
|---|---|---|---|
| Sample | DSPC | Lipid composition/mol % | (incorporated by reference) |
| baseline | 10 mol % | Ionizable lipid | IL1, WO 2022/246571; nor-MC3 |
| | | (IL)1:DSPC:Chol:PEG₂₀₀₀- | |
| | | DMG (50:10:38.5:1.5) | |
| lcLNP ™ | 50 mol % | Ionizable lipid (IL) | IL2, PCT/CA2023/051727 |
| (PEG-less) | | 2:DSPC:Chol | (compound 24) |
| | | (28.15:50:21.85) | |

The baseline formulation and the PEG-less lcLNP™ formulations in Table 1 above had favourable physiochemical characteristics. The results are summarized below in Table 2.

TABLE 2
Physiochemical characteristics of the baseline and lcLNP ™ of Table 1

| | | Encapsulation | Size Z-Average | Size number- | |
|---|---|---|---|---|---|
| Group | N/P | % | (nm) | average (nm) | PDI |
| baseline | 6 | 97 | 71.7 | 55.6 | 0.163 |
| lcLNP ™ | 9 | 97 | 106.3 | 76.9 | 0.193 |
| (PEG-less) | | | | | |

Live/dead staining and measurement of eGFP using flow cytometry was employed assessed for the CD8+ T cells. The eGFP was measured at 48 hours following dose titration (1, 2, 4, 6 and 8 μg) of the baseline and the lcLNP™.

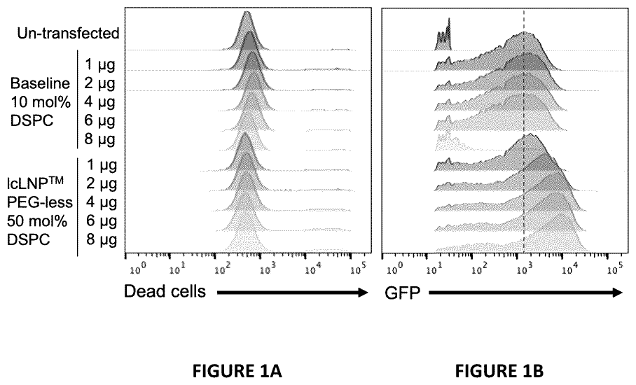

The results for the live/dead staining and mRNA expression of CD8+ T cells are presented in FIGS. 1A and 1B.

The results in FIGS. 1A and 1B show that at 48 hours post-addition of the LNPs at the indicated doses, the T cell samples treated with the GFP mRNA-containing lcLNP™ having 50 mol % DSPC had significantly more cells positive for GFP over the baseline formulation with only 10 mol % DSPC (FIG. 1B; see peak shift of lcLNP™ group).

A further surprising observation is that the lcLNP™ formulations lacking PEG outperformed the PEG-containing baseline formulation (FIG. 1i). In addition, as shown in FIG. 1B, at the 8 μg dose for GFP expression, the baseline LNP containing PEG exhibited a left shift relative to the other doses tested, whereas the PEG-less lcLNP™ at the same dose exhibited no such shift in expression. This result may suggest that the baseline LNP containing PEG is more toxic at this dose relative to the PEG-less LNP.

### Example 2: Gene Editing Efficacy Targeting the TRAC Locus Using High DSPC LNPs Relative to a Baseline Formulation

T cell receptor α constant (TRAC) gene editing knock-out (KG) efficiency using Cas9/CRSPR was next investigated comparing the baseline LNP and lcLNP™ with no PEG in primary CD8+ T cells. Gene knock-out of the TRAC gene locus encoding for the T cell receptor was of interest since it can be targeted for integration of CAR transgenes or to produce allogeneic T cells.

The following LNP formulations (reported in mol %) encapsulating mRNA encoding Cas9 and TRAC gRNA or scrambled gRNA were compared in this example for TRAC gene knock-out efficiency. Baseline LNPs with norMC3 and ionizable lipid 2 (baseline LNPs A and B) were compared to PEG-less lcLNP™ formulations with 50 mol DSPC with ionizable lipid 2 (LNP C and D). The baseline LNP contained Cas9/TRAC gRNA (LNPs A and B) and the PEG-less lcLNP™ formulations contained Cas9/TRAC gRNA (LNP C) or scrambled gRNA (LNP D).

TABLE 3
LNP formulations analyzed for TRAC gene editing knock-out efficiency

| | | | | Ionizable lipid (IL) | |
|---|---|---|---|---|---|
| | | Percent | | citation (incorporated | |
| LNP | Sample | DSPC | Lipid composition/mol % | by reference) | Cargo |
| A | baseline | 10 mol % | Ionizable lipid | IL1, WO 2022/246571; | Cas9/TRAC |
| | IL1 | | 1:DSPC:Chol:PEG₂₀₀₀- | nor-MC3 | gRNA |
| | | | DMG (50:10:38.5:1.5) | | |
| B | baseline | 10 mol % | Ionizable lipid 2 | IL2, | Cas9/TRAC |
| | IL2 | | (IL2):DSPC:Chol:PEG₂₀₀₀- | PCT/CA2023/051727 | gRNA |
| | | | DMG (50:10:38.5:1.5) | (compound 24) | |
| C | lcLNP ™ | 50 mol % | Ionizable lipid | IL2, | Cas9/TRAC |
| | (PEG- | | 2:DSPC:Chol | PCT/CA2023/051727 | gRNA |
| | less), IL2 | | (28.15:50:21.85) | (compound 24) | |
| D | lcLNP ™ | 50 mol % | Ionizable lipid | IL2, | Cas9/SCR |
| | (PEG-less) | | 2:DSPC:Chol | PCT/CA2023/051727 | gRNA |
| | scramble, | | (28.15:50:21.85) | (compound 24) | (scrambled) |
| | IL2 | | | | |

The Opattro™-type formulation (baseline) and the PEG-less lcLNP™ formulations in Table 3 above had favourable physiochemical characteristics. The results are summarized below in Table 4.

TABLE 4
Physiochemical characteristics of the baseline
and lcLNP ™ of Table 1

| | | | | Size Z- | Size | |
|---|---|---|---|---|---|---|
| | | | Encap- | Aver- | number- | |
| | | | sulation | age | average | |
| LNP | Sample | N/P | % | (nm) | (nm) | PDI |
| A | baseline IL1 | 6 | 98 | 74.6 | 58.4 | 0.035 |
| B | baseline IL2 | 6 | 94 | 87.0 | 66.0 | 0.069 |
| C | lcLNP ™ | 9 | 99 | 94.3 | 68.5 | 0.141 |
| | (PEG-less), | | | | | |
| | IL2 | | | | | |
| D | lcLNP ™ | 9 | 99 | 91.7 | 68.5 | 0.136 |
| | (PEG-less) | | | | | |
| | scramble, | | | | | |
| | IL2 | | | | | |

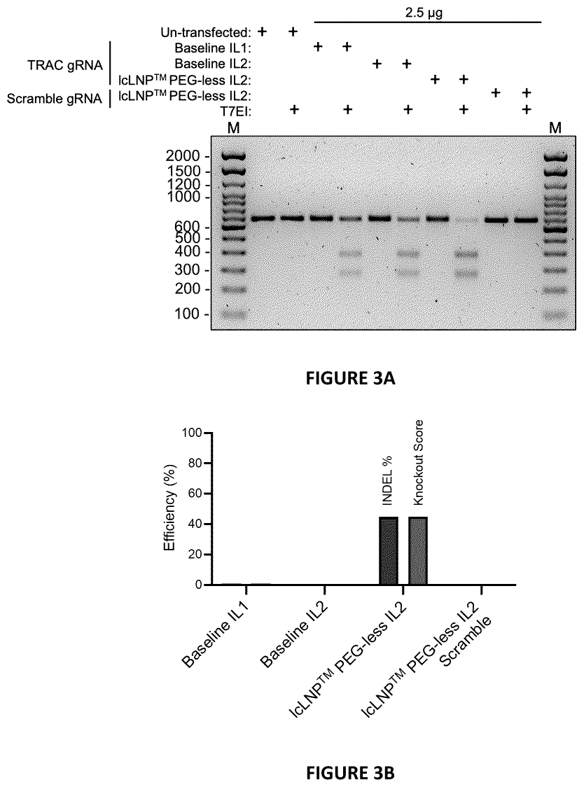

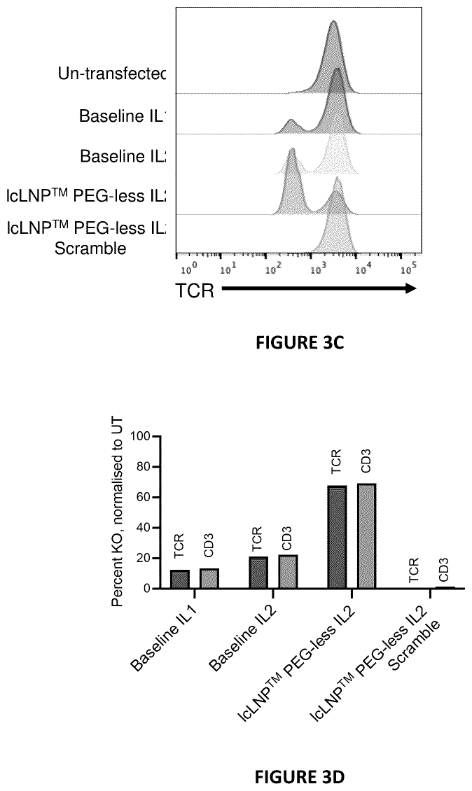

Primary human CD8+ T-cells were treated with the LNPs of Table 4. A T7 endonuclease detection assay (see Materials and Methods) was used to assess genome editing of the TRAC locus of the T-cells and the results are presented in the gel of FIG. 3A. The gel provides a measure of the cleaved heteroduplexes, which in turn corresponds to the level of indel (editing) activity. As can be seen, after the addition of the T7 endonuclease (T7EI), genomic DNA treated with the LNPs was digested and appears as three distinct bands (FIG. 3A, baseline LNP and lcLNP™ PEG-less), indicative of editing of the locus, while the control and lcLNP™ with scrambled gRNA remained undigested.

FIG. 3B is a graph showing gene editing efficiency (%) for Baseline IL1; BaselineIL2; lcLNP™ PEG-less IL2; and lcLNP™ PEG-less IL2 scramble. Surprisingly, only the lcLNP™ PEG-less IL2 showed any significant gene editing measured as INDEL % and knockout score. Both baseline LNP and lcLNP™ PEG-less IL2 scramble formulations had zero INDEL % and knockout scores.

FIG. 3C shows flow cytometry results measuring T cell receptor (TCR) 8 days post-LNP treatment comparing LNPs A-D of Table 4 above. Surprisingly, only the lcLNP™ PEG-less sample with intact TRAC gRNA exhibited a shift in the peak indicative of cells with lower T cell receptor count. The un-transfected sample, baseline LNP samples with PEG and lcLNP™ PEG-less with scrambled TRAC gRNA did not show any shift in the peaks indicative of lower TCR count.

FIG. 3D shows percentage knock-out for each LNP sample of Table 4 normalized to the untreated sample. Only the lcLNP™ PEG-less sample with intact TRAC gRNA exhibited significant knock-out of TCR and CD3 expression (>60%). The remaining PEG-containing baseline LNPs had knock-out percentages of around 20% or less relative to the untreated sample.

### Example 3: Characteristics and Efficacy of PEG-Less lcLNP™ in Primary Human CD4+ and CD8+ T Cells

The PEG-less lcLNP™ sample C of Table 3 of Example 2 having ionizable lipid 2:DSPC:Chol (28.15:50:21.85) was analyzed for T cell receptor (TCR) expression by flow cytometry 8 days post LNP treatment with CD4+ and CD8+ cells at a dose of 2.5 μg.

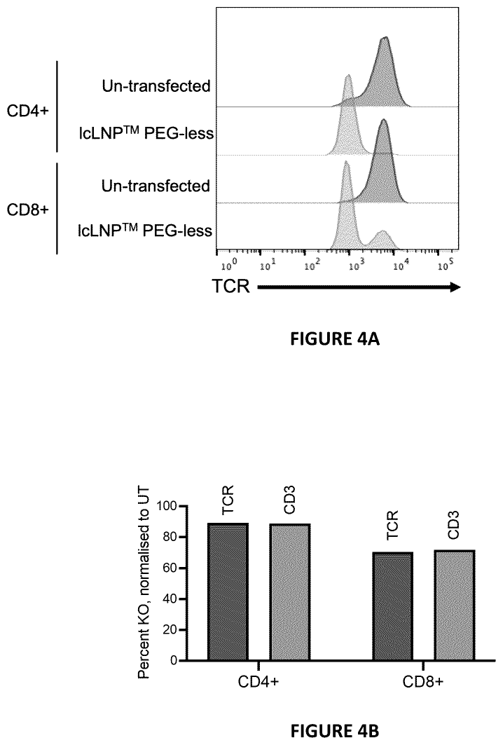

As can be seen in FIG. 4A, the PEG-less lcLNP™ with Cas9 and TRAC gRNA showed reductions in TCR signal for both CD4+ and CD8+ human T cells. The percentage knock-out (KO) of TCR and CD3 relative to the untreated cells was around 90% for CD4+ cells and around 70% for CD8+ cells (FIG. 4B).

### Example 4: In Vivo mRNA Expression of lcLNP™ in Bone Marrow and Blood T Cells

Given the positive ex vivo results with lcLNP™ in the previous examples, a variety of lcLNP™ formulations with 50 mol % DSPC and with various ionizable cationic lipids were compared to the baseline LNP with 10 mol % DSPC for in vivo expression in T cells of the bone marrow and blood using flow cytometry.

The LNPs in this example had the following composition:

TABLE 5
LNPs analyzed for eGFP expression in vivo using flow cytometry

| | | Percent | Lipid composition/molar | Ionizable lipid (IL) citation |
|---|---|---|---|---|
| LNP | Sample | DSPC | ratios | (incorporated by reference) |
| A | baseline IL1 | 10 | Ionizable lipid 1 | IL1, WO 2022/246571; nor- |
| | | mol % | (IL1):DSPC:Chol:PEG₂₀₀₀- | MC3 |
| | | | DMG (50:10:38.5:1.5) | |
| B | lcLNP ™ IL3 | 50 | Ionizable lipid 3 | IL3, PCT/CA2023/051727 |
| | | mol % | (IL3):DSPC:Chol:PEG₂₀₀₀- | (compound 5) |
| | | | DMG (27.4:50:21.1:1.5) | |
| C | lcLNP ™ IL4 | 50 | Ionizable lipid | IL4, PCT/CA2023/051727 |
| | | mol % | 4:DSPC:Chol:PEG₂₀₀₀-DMG | (compound 34) |
| | | | (27.4:50:21.1:1.5) | |
| D | lcLNP ™ IL5 | 50 | Ionizable lipid | IL5, U.S. provisional patent |
| | | mol % | 5:DSPC:Chol:PEG₂₀₀₀- | application No. 63/517,628 filed |
| | | | DMG (27.4:50:21.1:1.5) | on Aug. 4, 2023 (compound |
| | | | | 9) |
| E | lcLNP ™ IL2 | 50 | Ionizable lipid | IL2, PCT/CA2023/051727 |
| | | mol % | 2:DSPC:Chol:PEG₂₀₀₀- | (compound 24) |
| | | | DMG (27.4:50:21.1:1.5) | |
| F | lcLNP ™ IL6 | 50 | Ionizable lipid | IL6, PCT/CA2023/051727 |
| | | mol % | 6:DSPC:Chol:PEG₂₀₀₀-DMG | (compound 22) |
| | | | (27.4:50:21.1:1.5) | |
| G | lcLNP ™ IL7 | 50 | Ionizable lipid | IL7, PCT/CA2023/051727 |
| | | mol % | 7:DSPC:Chol:PEG₂₀₀₀-DMG | (compound 31) |
| | | | (27.4:50:21.1:1.5) | |

In a first study, LNPs A-E in Table 5 above were assessed for T cell eGFP expression in vivo. The physiochemical data in FIG. 5A shows that the LNP sizes, polydispersity index (PDI) and encapsulation percentages of LNPs A-E were all within acceptable ranges. Flow cytometry results of bone marrow T cells are shown in FIG. 5B. FIG. 5C shows CD4+ and CD8+ T cell subpopulations. In each case, the LNPs with 50 mol % DSPC (LNPs B-E) had higher 00 eGFP+ cells than the baseline LNP (LNP A) with 10 mol % DSPC. Similar trends were observed with the same formulations in blood T cells (FIGS. 5D and 5E).

In a second study, LNPs E, F and G in Table 5 above were assessed for eGFP expression in vivo. LNPs F and G were the same formulations as in the first study (ionizable lipid 7:DSPC:Chol:PEG₂₀₀₀-DMG (27.4:50:21.1:1.5 mol:mol)), but contained ionizable lipids 6 and 7 (see Table 5 above). FIG. 6A shows that the LNP sizes, polydispersity index (PDI) and encapsulation percentages of these LNPs were all within acceptable ranges. Flow cytometry results of bone marrow T cells and CD4+ and CD8+ T cell subpopulations are shown in FIG. 6B and FIG. 6C respectively. Similar to the results in the first study, in each case, the LNPs with 50 mol % DSPC (LNPs E, F and G) had higher % eGFP+ T cells than the baseline LNP (LNP A) in bone marrow.

### Example 5: Ex Vivo LNP-Mediated Knock-In of Primary T Cells

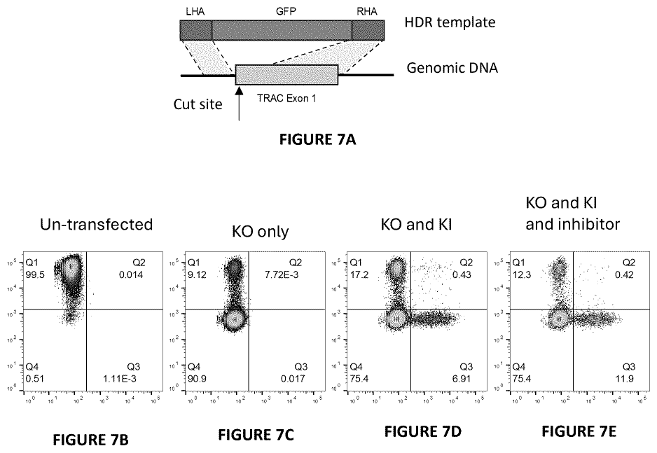

Given the successful knock-out of the TRAC locus achieved in the previous examples, the inventors next investigated LNP-mediated knock-in of eGFP in the TRAC locus of CD4+ T cells. The LNP formulations examined were as follows: lcLNP™ PEG-less:Ionizable lipid 2:DSPC:Chol (28.15:50:21.85) The LNP cargo was Cas9 mRNA:TCR gRNA (1:1 wt:wt) for knock-out only (control) or Cas9 mRNA:TCR gRNA:GFP homology directed repair (HDR) DNA (1:1:3 wt:wt) for the knock-out/knock-in studies. The cut site in the TRAC exon 1 is depicted in FIG. 7A, as well as the HDR template comprising a sequence encoding GFP flanked by left homology arm (LHA) and right homology arm (RHA) sequences. The knock-out and knock-out/knock-in studies were carried out as set forth in the Materials and Methods.

The results are shown in FIGS. 7B-E. Un-transfected T cells were primarily of the wild-type phenotype as measured by flow cytometry (FIG. 7B). The T cells treated with LNPs having only knock-out cargo, Cas9 mRNA:TCR gRNA, exhibited 91% knock out (FIG. 7C). The knock-out/knock-in sample (KO+KI) treated with LNPs having Cas9 mRNA:TCR gRNA:GFP HDR DNA exhibited 75% knock-out and 7% knock-in (FIG. 7D), while the same knock-out/knock-in sample treated with NHEJ inhibitor M3814 exhibited 75% knock-out and 11% knock-in (FIG. 7E).

The examples are intended to illustrate the preparation of specific lipid nanoparticle preparations and properties thereof but are in no way intended to limit the scope of the invention.

The article “a” or “an” as used herein is meant to include both singular and plural, unless otherwise indicated.

1. A method for delivery of nucleic acid to a T cell to produce a modified T cell, the method comprising contacting a lipid nanoparticle encapsulating the nucleic acid with the T cell ex vivo or in vivo, thereby causing cellular uptake of the nucleic acid, the lipid nanoparticle having between 30 mol % and 70 mol % of a neutral or zwitterionic amphipathic lipid having a net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid modifies the T cell by: (i) altering expression of a protein, polypeptide or peptide in the T-cell; and/or (ii) expressing an endogenous or exogenous protein, polypeptide or peptide in the T cell, thereby producing the modified T cell, wherein the modified T cell thereby produced provides a therapeutic, prophylactic or ameliorative effect in vivo.

2. The method of claim 1, wherein the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

3. The method of claim 1, wherein the contacting is in vivo and the T cell is in the blood, spleen or bone marrow of a subject.

4. The method of claim 1, wherein the nucleic acid is mRNA for expressing an endogenous or exogenous protein, polypeptide or peptide in the T-cell.

5. The method of claim 4, wherein the endogenous or exogenous protein, polypeptide or peptide is a chimeric antigen receptor.

6. The method of claim 1, wherein the nucleic acid is part of a nucleic acid editor.

7. The method of claim 6, wherein the nucleic acid editor is a Cas-based editor, transcription activator-like effector nuclease (TALEN), megaTAL, zinc finger nuclease (ZFN), Adenosine Deaminase Acting on RNA (ADAR), prime editor, base editor, epigenetic editor, transposase, meganuclease, ARCUS gene editing system or a combination thereof.

8. The method of claim 7, wherein the Cas-based editor is CRISPR and comprises a guide RNA and an mRNA or vector DNA coding for a Cas nuclease.

9. The method of claim 1, wherein the lipid nanoparticle is for treating a disease or disorder that is an immunological disease or disorder.

10. The method of claim 1, wherein the lipid nanoparticle is for treating a disease or disorder that is a cancer.

11. The method of claim 10, wherein the cancer is a haematological cancer.

12. A lipid nanoparticle comprising an encapsulated nucleic acid for ex vivo or in vivo delivery to a T cell to produce a modified T cell having a therapeutic, prophylactic or ameliorative effect in vivo, the lipid nanoparticle having between 30 mol % and 70 mol % of neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid is for modifying the T cell by: (i) altering expression of an endogenous protein, polypeptide or peptide in the T-cell; and/or (ii) expressing an endogenous or exogenous protein, polypeptide or peptide in the T cell,
wherein, the lipid nanoparticle, when encapsulating eGFP, exhibits at least a 10% increase in expression of eGFP in CD4 or CD8 T cells of the blood or bone marrow relative to a baseline formulation having 50/10/38.5/1.5 mol/mol ionizable cationic lipid, DSPC, cholesterol and PEG-lipid measured at 24 hours post-injection to a mouse model.

13. A lipid nanoparticle comprising an encapsulated nucleic acid for ex vivo or in vivo delivery to a T cell to produce a modified T cell having a therapeutic, prophylactic or ameliorative effect in vivo, the lipid nanoparticle having between 30 mol % and 70 mol % of neutral lipid or zwitterionic amphipathic lipid having a neutral or net-neutral charge at physiological pH, an ionizable cationic lipid, and optionally a sterol, wherein the lipid nanoparticle is substantially uncharged at physiological pH and has an apparent pKa of between 6.0 and 7.5, wherein the nucleic acid is for modifying the T cell and wherein the lipid nanoparticle has less than 1.5 mol % PEG-lipid.

14. The lipid nanoparticle of claim 12, wherein the neutral or zwitterionic lipid is a phospholipid having a choline head group and is selected from distearoylphosphatidylcholine (DSPC), dimyristoylphosphatidylcholine (DMPC) and/or dipalmitoyl-phosphatidylcholine (DPPC).

15. The lipid nanoparticle of claim 12, wherein the lipid nanoparticle is for modifying the T cell in vivo in a subject's blood, spleen or bone marrow.

16. The lipid nanoparticle of claim 12, wherein nucleic acid is for expressing an endogenous or exogenous protein or peptide in the T-cell.

17. The lipid nanoparticle of claim 16, wherein the endogenous or exogenous protein or peptide is a chimeric antigen receptor.

18. The lipid nanoparticle of claim 12, wherein the nucleic acid is part of a nucleic acid editor.

19. The lipid nanoparticle of claim 18, wherein the nucleic acid editor is a Cas-based editor, transcription activator-like effector nuclease (TALEN), megaTAL, zinc finger nuclease (ZFN), Adenosine Deaminase Acting on RNA (ADAR), prime editor, base editor, epigenetic editor, transposase, meganuclease, ARCUS gene editing system or a combination thereof.

20. The lipid nanoparticle of claim 19, wherein the Cas-based editor is CRISPR and comprises a guide RNA and an mRNA or vector DNA coding for a Cas nuclease.

21. (canceled)

22. (canceled)

23. (canceled)

24. (canceled)

25. An ex vivo CD4 or CD8 T cell preparation comprising the lipid nanoparticle of claim 13.
